(19) United States
(12) Patent Application Publication
(10) Pub. No.: US 2026/0285808 A1
(43) Pub. Date: Sep. 24, 2026

(54) ANTIBIOTICS COMPRISING LPXH-TARGETING COMPOUNDS AND METHODS OF MAKING AND USING THE SAME

(71) Applicant: Duke University, Durham, NC (US)

(72) Inventors: Pei Zhou, Durham, NC (US); Jiyong Hong, Durham, NC (US)

(21) Appl. No.: 19/471,912

(22) PCT Filed: Apr. 3, 2024
(86) PCT No.: PCT/US2024/022884

§ 371 Date: Oct. 2, 2025

Related U.S. Application Data
(60) Provisional application No. 63/456,606, filed on Apr. 3, 2023.
(60) Provisional application No. 63/524,037, filed on Jun. 29, 2023.

(51) Int. Cl. C07D 209/08; A61K 31/495; A61K 31/496; A61P 31/04; C07D 249/04; C07D 295/26; C07D 401/12

(52) U.S. Cl. CPC C07D 209/08; A61K 31/495; A61K 31/496; A61P 31/04; C07D 249/04; C07D 295/26; C07D 401/12

(57) ABSTRACT

Disclosed herein are novel antibiotic compositions and methods of making and using the same. The antibiotic may comprise LPXH-targeting compounds.

### I. CROSS-REFERENCE TO RELATED APPLICATIONS

This application claims priority to U.S. Provisional Application No. 63/456,606 filed on Apr. 3, 2023 and U.S. Provisional Application No. 63/524,037 filed on Jun. 29, 2023, which are incorporated herein in their entirety.

### II. STATEMENT REGARDING FEDERALLY SPONSORED RESEARCH OR DEVELOPMENT

This invention was made with Government support under Federal Grant nos. A1139216 and GM115355 awarded by the National Institutes of Health. The Federal Government has certain rights to this invention.

### III. BACKGROUND

Gram-negative bacterial pathogens cause many life-threatening illnesses, including pneumonia and bloodstream, wound, and surgical site infections in healthcare and community settings. Antibiotics are essential medications for the treatment of Gram-negative bacterial infection. Unfortunately, as more and more Gram-negative bacteria are becoming resistant to nearly all antibiotics, patients are left with fewer effective treatment options. According to the report by Centers for Disease Control and Prevention on antibiotic resistance threats in the United States, over half of the eighteen antibiotic-resistant threats are from Gram-negative bacteria. In spite of these looming public health concerns, there has been little activity in the antibiotic development pipeline, with no antibiotic with a novel mode of action against Gram-negative bacteria discovered during the past five decades. Hence, there is an urgent need for new antibiotics to treat bacterial infections caused by multidrug-resistant Gram-negative bacteria.

Since the outer membrane of Gram-negative bacteria is required for bacterial viability and serves as a permeability barrier to prevent toxic compounds from entering the cell, disrupting its biogenesis presents an attractive strategy for antibacterial drug discovery. A distinctive feature of the outer membrane of Gram-negative pathogens is the presence of lipopolysaccharide (LPS) or lipooligosaccharide (LOS) extruding from the outer monolayer of the outer membrane. Lipid A is the hydrophobic membrane anchor of LPS/LOS and the active component of the bacterial endotoxin that causes Gram-negative septic shock during bacterial infection. Its constitutive biosynthesis via the Raetz pathway is required for bacterial viability and fitness in the human host (FIG. 1A). LpxH is an essential, late-stage lipid A enzyme that hydrolyzes the pyrophosphate group of UDP-2,3-diacylglucosamine (UDP-DAGn) to yield 2,3-diacylglucosamine 1-phosphate (lipid X) and UMP. It is found in ~70% of Gram-negative bacteria and all of the World Health Organization priority Gram-negative pathogens, whereas in the remaining bacterial species, the same chemical reaction is catalyzed by LpxI and LpxG, the functional paralogs of LpxH. Biochemical studies have established LpxH as a member of the calcineurin-like phosphoesterases (CLPs), which requires detergent and a di-manganese cluster for full enzymatic activity. Structural analyses of H. influenzae LpxH, P. aeruginosa LpxH, E. coli LpxH, and most recently K. pneumoniae LpxH (KpLpxH) have collectively revealed the presence of a conserved core architecture of CLPs with a unique insertion lid above it. The active site, situating between the lid domain and the core CLP architecture, contains a di-manganese clusterthat is chelated by residues from the signature metal chelating motifs of CLP enzymes.

LpxH is a uniquely attractive antibiotic target for multidrug-resistant Gram-negative bacteria, as inhibition of LpxH not only disrupts lipid A biosynthesis, but also accumulates toxic lipid A intermediates in the bacterial inner membrane. In 2015, AstraZeneca reported a sulfonyl piperazine compound, dubbed AZ1 (1, FIG. 1B), as the first LpxH inhibitor. AZ1 (1) displays weak antibiotic activity against WT K. pneumoniae and E. coli strains with a compromised outer membrane (yhjD*ΔkdtA) orwith an efflux pump deletion (ΔtoIC).

### IV. BRIEF DESCRIPTION OF THE FIGURES

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIG. 1A is a schematic showing the disruption of lipid A biosynthesis by LpxH inhibitors. Specifically showing a schematic illustration of the Raetz pathway of lipid A biosynthesis in E. coli. LpxH and its functional orthologs, LpxI and LpxG, are labeled in purple and green, respectively.

FIG. 1B shows chemical structures of the LpxH inhibitors: AZ1 (1), JH-LPH-33 (2), and JH-LPH-41 (3) in accordance with one embodiment of the present disclosure.

FIG. 2A is a schematic showing the design of new LpxH inhibitors in accordance with embodiments of the present disclosure. The N-acyl chain extension of JH-LPH-41 (3) (PDB: 6WII) overlaps with the hexose ring shared by the LpxH product and the substrate (lipid X and UDP-DAGn, respectively) (PDB: 6PH9).

FIG. 2B shows general formulae of newly designed LpxH inhibitors with an acyl-hydroxamate extension from the indoline (4) and aniline (5) scaffolds.

FIGS. 3A-3E are graphs and schematic illustrations showing the inhibition of KpLpxH by JH-LPH-45 in accordance with one embodiment of the present disclosure.

FIG. 3A is a ribbon diagram of KpLpxH/JH-LPH-45 complex. KpLpxH is colored in rainbow, with the N-terminus in blue and C-terminus in red. JH-LPH-45 and the di-manganese cluster are shown in the stick and sphere models, respectively. The purple mesh represents the 2mFo-DFc map of JH-LPH-45 at 10.

FIG. 3B is an enlarged view of JH-LPH-45. Superimposition of the KpLpxH/JH-LPH-45 complex with the previously reported KpLpxH/lipid X complex (PDB: 6PH9) shows that the compound competes with the 2-N-acyl chain of lipid X, but with its acyl-hydroxamate group chelating the active site di-manganese cluster.

FIG. 3C shows interactions between JH-LPH-45 and LpxH residues. The interaction map was generated by LigPlot+.

FIG. 3D shows metal chelation geometry. Distances between the atoms in the hydroxamate group and the di-manganese cluster are labeled with dashed lines.

FIG. 3E is an IC₅₀ curve of the KpLpxH inhibition by JH-LPH-45 in accordance with embodiments of the present disclosure. Error bars represent the standard error of measurement (SEM, n=3).

FIGS. 4A-4E are graphs and schematics showing the inhibition of KpLpxH by JH-LPH-50 in accordance with one embodiment of the present disclosure.

FIG. 4A is ribbon diagram of the KpLpxH/JH-LPH-50 complex. KpLpxH is colored in rainbow, with the N-terminus in blue and C-terminus in red. JH-LPH-50 and the di-manganese cluster are shown in the stick and sphere models, respectively. The purple mesh represents the 2mFo-DFc map of JH-LPH-50 at 0.8 a with JH-LPH-50 shown in the stick model.

FIG. 4B is a pictorial illustration showing an enlarged view of JH-LPH-50. Superimposition of the KpLpxH/JH-LPH-50 complex with the previously reported KpLpxH/lipid X complex (PDB: 6PH9) shows that the compound competes with the 2-N-acyl chain of lipid X, but with its acyl-hydroxamate group chelating the active site di-manganese cluster.

FIG. 4C shows interactions between JH-LPH-50 and LpxH residues. The interaction map was generated by LigPlot+.

FIG. 4D shows metal chelation geometry. Distances between the atoms in the hydroxamate group and the di-manganese cluster are labeled with dashed lines.

FIG. 4E is an IC₅₀ curve of the KpLpxH inhibition by JH-LPH-50. Error bars represent the standard error of measurement (SEM, n=3).

### V. BRIEF SUMMARY

Other aspects and iterations of the present disclosure are detailed below.

In some aspects, the present disclosure is directed to a compound of formula (I), or a pharmaceutically acceptable salt thereof:

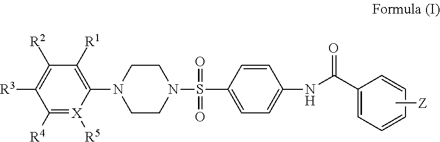

wherein
X is carbon or nitrogen;
R¹-R⁵ are each independently hydrogen, hydroxyl, halogen, CF₃, C1-C4 alkyl, or substituted C1-C4 alkyl, or absent, wherein substituted C1-C4 alkyl is optionally halogen substituted C1-C4 alkyl; and
Z is hydrogen, an alkylsulfonamide, or a substituted alkylsulfonamide attached to the aryl by either N or S.

In some aspects, the halogen is fluorine, chlorine, or bromine.

In some aspects, the halogen is chlorine.

In some aspects, R² and R⁴ are independently halogen or CF₃.

In some aspects, R² and R⁴ are independently chlorine or CF₃.

In some aspects, R¹ and R³ are hydrogen; X is nitrogen, and W is absent.

In some aspects, R¹ and R³ are hydrogen.

In some aspects, X is nitrogen and R⁵ is absent.

In some aspects, X is carbon and R⁵ is hydrogen.

In other aspects, the present disclosure is directed to a compound of formula (II), or a pharmaceutically acceptable salt thereof:

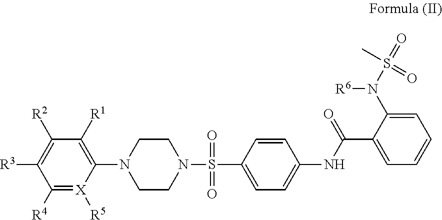

wherein
X is carbon or nitrogen,
R¹-R⁶ are each independently hydrogen, hydroxyl, halogen, CF₃, C1-C4 alkyl, or substituted C1-C4 alkyl, or absent, wherein substituted C1-C4 alkyl is optionally halogen substituted C1-C4 alkyl.

In some aspects, the halogen is fluorine, chlorine, or bromine.

In some aspects, the halogen is chlorine.

In some aspects, R² and R⁴ are independently halogen or CF₃.

In some aspects, R² and R⁴ are independently chlorine or CF₃.

In some aspects, R¹ and R³ are hydrogen, X is nitrogen, and R⁵ is absent.

In some aspects, R¹ and P³ are hydrogen.

In some aspects, X is nitrogen and R⁵ is absent.

In some aspects, X is carbon and P⁵ is hydrogen.

In some aspects, R⁶ is C1-C4 alkyl or substituted C1-C4 alkyl.

In some aspects, R⁶ is methyl.

In some aspects, the compound is

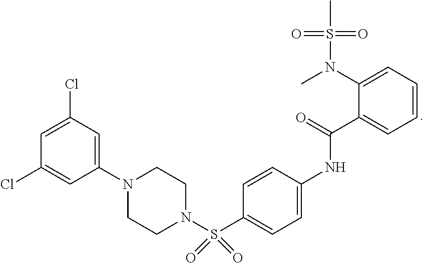 [SMILES: CN(c1ccccc1C(=O)Nc1ccc(S(=O)(=O)N2CCN(c3cc(Cl)cc(Cl)c3)CC2)cc1)S(C)(=O)=O]

In other aspects, the present disclosure is directed to a compound of formula (III), or a pharmaceutically acceptable salt thereof:

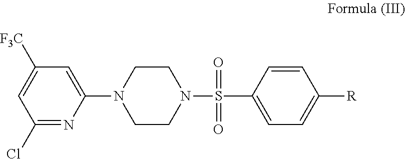

wherein
R is a substituted amide with formula —NHC(O)A; and
A is a substituted aryl.

In some aspects, the substituted aryl is

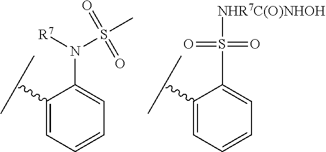

wherein
R⁷ is C1-C8 alkyl or R⁸C(O)NHOH; and
R⁸ is C1-C8 alkyl.

In some aspects, the compound is

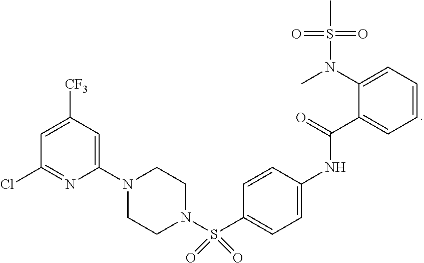 [SMILES: CN(c1ccccc1C(=O)Nc1ccc(S(=O)(=O)N2CCN(c3cc(C(F)(F)F)cc(Cl)n3)CC2)cc1)S(C)(=O)=O]

In some aspects, the compound is

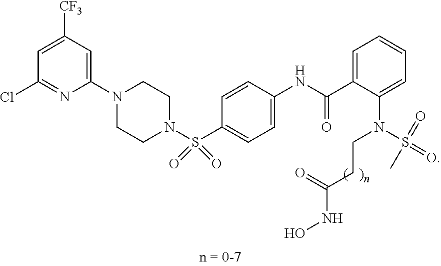

In some aspects, the compound is

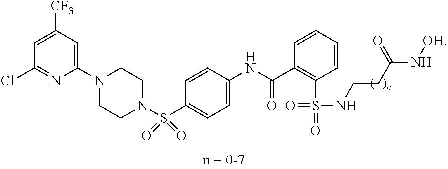

In yet other aspects, the present disclosure is directed a compound of formula (IV), or a pharmaceutically acceptable salt thereof:

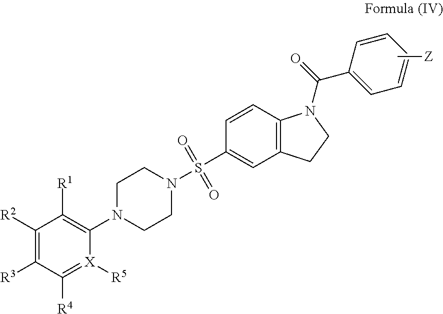

wherein
X is carbon or nitrogen;
R¹-R⁵ are each independently hydrogen, hydroxyl, halogen, CF₃, C1-C4 alkyl, or substituted C1-C4 alkyl, or absent, wherein substituted C1-C4 alkyl is optionally halogen substituted C1-C4 alkyl; and
wherein Z is an alkylsulfonamide attached to the aryl by either N or S.

In some aspects, the halogen is fluorine, chlorine, or bromine.

In some aspects, the halogen is chlorine.

In some aspects, R² and R⁴ are independently halogen or CF₃.

In some aspects, R² and R⁴ are independently chlorine or CF₃.

In some aspects, R¹ and R³ are hydrogen, X is nitrogen, and R⁵ is absent.

In some aspects, R¹ and R³ are hydrogen.

In some aspects, X is nitrogen and R⁵ is absent.

In some aspects, X is carbon and R⁵ is hydrogen.

In some aspects, the compound is

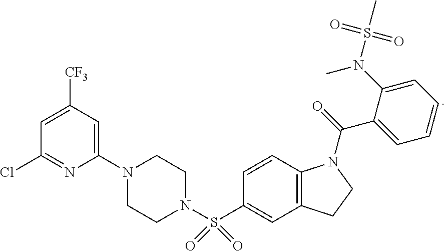 [SMILES: CN(c1ccccc1C(=O)N1CCc2cc(S(=O)(=O)N3CCN(c4cc(C(F)(F)F)cc(Cl)n4)CC3)ccc21)S(C)(=O)=O]

In some aspects, the present disclosure is directed to a compound, or a pharmaceutically acceptable salt thereof, wherein the is selected from the group consisting of

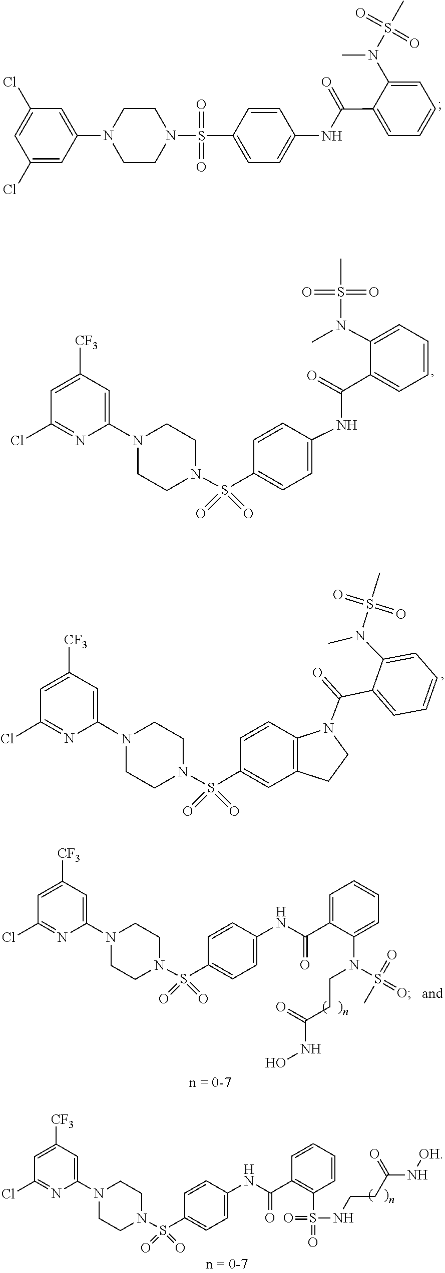

In yet other aspects, the present disclosure is directed to a pharmaceutical composition comprising the compound of any one of the claims above and a pharmaceutically acceptable carrier, diluent, and/or an excipient.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase ‘consisting essentially of’ limits the scope of a claim to the recited components in a composition or the recited steps in a method as well as those that do not materially affect the basic and novel characteristic or characteristics of the claimed composition or claimed method. The phrase ‘consisting of’ excludes any component, step, or element that is not recited in the claim. The phrase ‘comprising’ is synonymous with ‘including’, ‘containing’, or ‘characterized by’, and is inclusive or open-ended. ‘Comprising’ does not exclude additional, unrecited components or steps.

As used herein, when referring to any numerical value, the term ‘about’ means a value falling within a range that is ±10% of the stated value.

Ranges can be expressed herein as from ‘about’ one particular value, and/or to ‘about’ another particular value. When such a range is expressed, a further aspect includes from the one particular value and/or to the other particular value. Similarly, when values are expressed as approximations, by use of the antecedent ‘about,’ it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as ‘about’ that particular value in addition to the value itself. For example, if the value ‘10’ is disclosed, then ‘about 10’ is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms ‘optional’ or ‘optionally’ means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term ‘subject’ refers to the target of administration, e.g., a human being. The term ‘subject’ also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have cancer, be suspected of having cancer, or be at risk of developing cancer.

The term “disease” as used herein includes, but is not limited to, any abnormal condition and/or disorder of a structure or a function that affects a part of an organism. It may be caused by an external factor, such as an infectious disease (e.g., a bacterial infection), or by internal dysfunctions, such as cancer, cancer metastasis, and the like.

A “patient” refers to a subject afflicted with a disease or disorder (e.g., a hematological cancer). In an aspect, a patient can refer to a subject that has been diagnosed with or is suspected of having a disease or disorder such as a hematological cancer. In an aspect, a patient can refer to a subject that has been diagnosed with or is suspected of having a disease or disorder and is seeking treatment or receiving treatment for a disease or disorder (such as hematological cancer).

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician).

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having received one or more of the disclosed chimeric fusion proteins, the disclosed isolated nucleic acid molecules, the disclosed vectors, the disclosed cells, the disclosed pharmaceutical formulations, or a combination thereof). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to a native or control level (e.g., a subject not having received one or more of the disclosed chimeric fusion proteins, the disclosed isolated nucleic acid molecules, the disclosed vectors, the disclosed cells, the disclosed pharmaceutical formulations, or a combination thereof). In an aspect, the inhibition or reduction can be 0-25%, 25-50%, 50-75%, or 75-100% as compared to native or control levels. In an aspect, a native or control level can be a pre-disease or pre-disorder level (such as a pre-cancer state).

The words “treat” or “treating” or “treatment” include palliative treatment, that is, treatment designed for the relief of symptoms rather than the curing of the disease, pathological condition, or disorder (e.g., a hematological cancer); preventative treatment, that is, treatment directed to minimizing or partially or completely inhibiting the development of the associated disease, pathological condition, or disorder (e.g., a hematological cancer); and supportive treatment, that is, treatment employed to supplement another specific therapy directed toward the improvement of the associated disease, pathological condition, or disorder (e.g., a hematological cancer). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having cancer). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (e.g., a hematological cancer). For example, treating a disease or disorder can reduce one or more symptoms of a disease or disorder in a subject by 1%-100% as compared to a control (such as, for example, an individual not having cancer). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder (e.g., a hematological cancer). It is understood that treatment does not necessarily refer to a cure or complete ablation or eradication of a disease or disorder. However, in an aspect, treatment can refer to a cure or complete ablation or eradication of a disease or disorder (such as a hematological cancer).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as a hematological cancer) or related complication from progressing to that complication. In an aspect, preventing metastasis is intended.

As used herein, the terms “administering” and “administration” refer to any method of providing one or more of the disclosed chimeric fusion proteins, the disclosed isolated nucleic acid molecules, the disclosed vectors, the disclosed cells, the disclosed pharmaceutical formulations, or a combination thereof, or by one or more of the disclosed methods to a subject. Such methods are well known to those skilled in the art and include, but are not limited to, the following: oral administration, transdermal administration, administration by inhalation, nasal administration, topical administration, in utero administration, intratumoral administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of one or more of the disclosed chimeric fusion proteins, the disclosed isolated nucleic acid molecules, the disclosed vectors, the disclosed cells, the disclosed pharmaceutical formulations, or a combination thereof can comprise administration directly into the CNS or the PNS. Administration can be continuous or intermittent. Administration can comprise a combination of one or more routes.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “concurrently” means (1) simultaneously in time, or (2) at different times during the course of a common treatment schedule.

As used herein, “determining” can refer to measuring or ascertaining the presence and severity of a disease or disorder, such as, for example, a hematologic cancer. Methods and techniques used to determine the presence and/or severity of a disease or disorder are typically known to the medical arts. For example, the art is familiar with the ways to identify and/or diagnose the presence, severity, or both of a disease or disorder (such as, for example, cancer).

As used herein, “effective amount” and “amount effective” can refer to an amount that is sufficient to achieve the desired result such as, for example, the treatment and/or prevention of a disease or disorder (e.g., a hematological cancer) or a suspected disease or disorder (e.g., a hematological cancer). As used herein, the terms “effective amount” and “amount effective” can refer to an amount that is sufficient to achieve the desired an effect on an undesired condition (e.g., a hematological cancer). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount the disclosed chimeric fusion proteins, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof that (i) treats the particular disease, condition, or disorder (e.g., a hematological cancer), (ii) attenuates, ameliorates, or eliminates one or more symptoms of the particular disease, condition, or disorder e.g., a hematological cancer), or (iii) delays the onset of one or more symptoms of the particular disease, condition, or disorder described herein (e.g., a hematological cancer). The specific therapeutically effective dose level for any particular patient will depend upon a variety of factors including the disorder being treated and the severity of the disorder; one or more of the disclosed chimeric fusion proteins, the disclosed isolated nucleic acid molecules, the disclosed vectors, the disclosed cells, the disclosed pharmaceutical formulations, or any combination thereof employed; the disclosed methods employed; the age, body weight, general health, sex and diet of the patient; the time of administration; the route of administration; the rate of excretion of one or more of the disclosed chimeric fusion proteins, the disclosed isolated nucleic acid molecules, the disclosed vectors, the disclosed cells, the disclosed pharmaceutical formulations, or any combination thereof employed; the duration of the treatment; drugs used in combination or coincidental with the one or more of the disclosed chimeric fusion proteins, the disclosed isolated nucleic acid molecules, the disclosed vectors, the disclosed cells, the disclosed pharmaceutical formulations, or any combination thereof employed, and other like factors well known in the medical arts. For example, it is well within the skill of the art to start doses of the disclosed chimeric fusion proteins, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed chimeric fusion proteins, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a hematological cancer.

As used herein, “conjugate” or “conjugated” can be used to define the operative association of one disclosed component to another disclosed component. In an aspect, conjugated does not intend to refer solely to any type of operative association and is not particularly limited to chemical “conjugation”.

A “target” or “target antigen” is any molecule bound by a binding motif (e.g., a PS-expressing hematological cancer cell). A disclosed target can be cells and/or tissues in a subject.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order.

The use of the term “in combination” does not restrict the order in which therapies are administered to a subject. By way of non-limiting example, a first therapy (e.g., one or more of the disclosed chimeric fusion proteins, the disclosed isolated nucleic acid molecules, the disclosed vectors, the disclosed cells, the disclosed pharmaceutical formulations, or a combination thereof) can be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy to a subject having or diagnosed with hematological cancer.

### B. Sulfonyl Piperazine LpxH Inhibitors

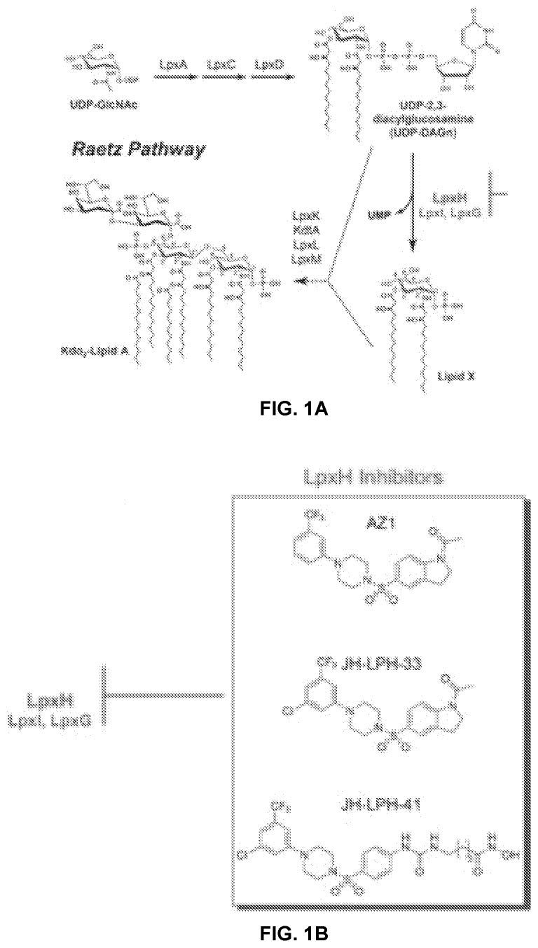

In some aspects, the present disclosure is directed to the use of AZ1 as a novel antibiotic targeting LpxH. In some aspects, the detailed structure-activity relationship of AZ1 (1) may be analyzed and to elucidate the molecular details of the LpxH-AZ1 interaction by X-ray crystallography and NMR. By harnessing the ligand dynamics information and structural insights, sulfonyl piperazine LpXH inhibitors may be designed. Examples of the LpXH inhibitors include but is not limited to JH-LPH-33 (2, FIG. 1B), an AZ1 analog with significantly enhanced potency in enzymatic assays and outstanding antibiotic activities in vitro. Compounds 1 and 2 may function by occupying the hydrophobic LpxH substrate chamber that accommodates the 2-N-acyl chain of the substrate, but they do not reach into the active site of LpxH. In some aspects, the present disclosure if directed towards the development of JH-LPH-41 (3, FIG. 1B) that harbors an extended N-acyl chain reaching into the active site of LpxH. Despite these efforts, no LpxH inhibitor described to date chelates the di-manganese cluster in the active site of LpxH.

The present disclosure is directed to sulfonyl piperazine LpxH inhibitors.

The present disclosure is directed to a compound of formula (I), or a pharmaceutically acceptable salt thereof:

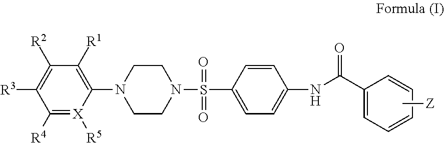

wherein
X₁-X₅ are each independently carbon or nitrogen;
R¹-R¹ are each independently hydrogen, hydroxyl, halogen, CF₃, C1-C4 alkyl, or substituted C1-C4 alkyl, or absent, wherein substituted C1-C4 alkyl is optionally halogen substituted C1-C4 alkyl; and
Z is hydrogen, an alkylsulfonamide, or a substituted alkylsulfonamide attached to the aryl by either N or S.

In some embodiments, the halogen is fluorine, chlorine, or bromine.

In some embodiments, the halogen is chlorine.

In some embodiments, R² and R⁴ are independently halogen or CF₃.

In some embodiments, R² and R⁴ are independently chlorine or CF₃.

In some embodiments, R¹ and R³ are hydrogen, X is nitrogen, and R is absent.

In some embodiments, R¹ and R³ are hydrogen.

In some embodiments, X is nitrogen and R⁵ is absent.

In some embodiments, X is carbon and R⁵ is hydrogen.

The present disclosure is directed to a compound of formula (II), or a pharmaceutically acceptable salt thereof:

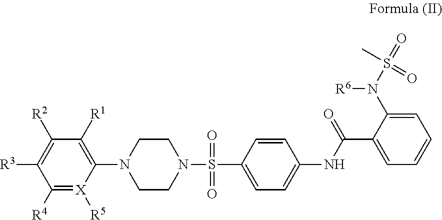

X is carbon or nitrogen,
R¹-R⁶ are each independently hydrogen, hydroxyl, halogen, CF₃, C1-C4 alkyl, or substituted C1-C4 alkyl, or absent: wherein substituted C1-C4 alkyl is optionally halogen substituted C1-C4 alkyl.

In some embodiments, the halogen is fluorine, chlorine, or bromine.

In some embodiments, the halogen is chlorine.

In some embodiments, R² and R⁴ are independently halogen or CF³.

In some embodiments, R² and R⁴ are independently chlorine or CF₃.

In some embodiments, R¹ and R³ are hydrogen, X is nitrogen, and R⁵ is absent.

In some embodiments, R¹ and R³ are hydrogen.

In some embodiments, X is nitrogen and R⁵ is absent.

In some embodiments, X is carbon and R⁵ is hydrogen.

In some embodiments, R⁶ is C1-C4 alkyl or substituted C1-C4 alkyl.

In some embodiments, R⁶ is methyl.

In some embodiments, the compound is

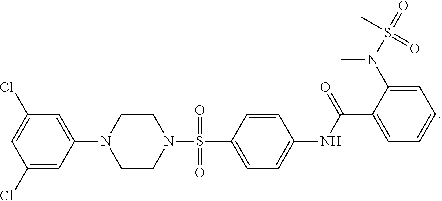 [SMILES: CN(c1ccccc1C(=O)Nc1ccc(S(=O)(=O)N2CCN(c3cc(Cl)cc(Cl)c3)CC2)cc1)S(C)(=O)=O]

The present disclosure is directed to a compound of formula (III), or a pharmaceutically acceptable salt thereof:

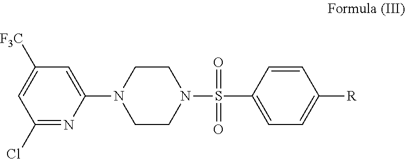

wherein
R is a substituted amide with formula —NHC(O)A; and
A is a substituted aryl.

In some embodiments, the substituted aryl is

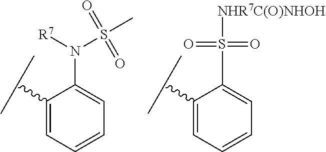

wherein
R⁷ is C1-C8 alkyl or R⁸C(O)NHOH; and
R⁸ is C1-C8 alkyl.

In some embodiments, the compound is

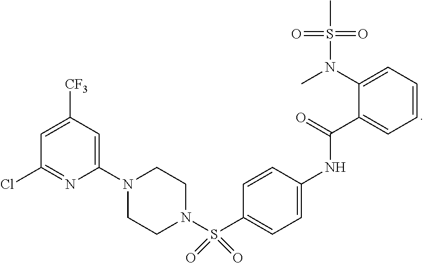 [SMILES: CN(c1ccccc1C(=O)Nc1ccc(S(=O)(=O)N2CCN(c3cc(C(F)(F)F)cc(Cl)n3)CC2)cc1)S(C)(=O)=O]

In some embodiments, the compound is

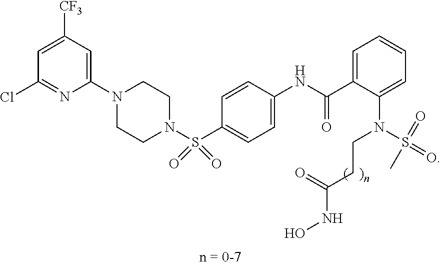

In some embodiments, the compound is

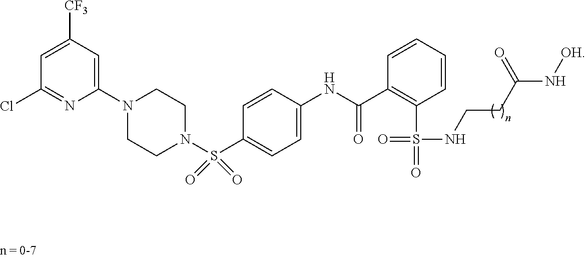

The present disclosure is directed to a compound of formula (IV),

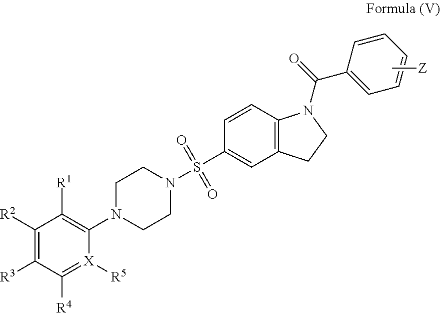

wherein
X is carbon or nitrogen;
R¹-R⁵ are each independently hydrogen, hydroxyl, halogen, CF₃, C1-C4 alkyl, or substituted C1-C4 alkyl, or absent, wherein substituted C1-C4 alkyl is optionally halogen substituted C1-C4 alkyl; and
wherein Z is hydrogen, an alkylsulfonamide, or a substituted alkylsulfonamide attached to the aryl by either N or S.

In some embodiments, the halogen is fluorine, chlorine, or bromine.

In some embodiments, the halogen is chlorine.

In some embodiments, R² and R⁴ are independently halogen or CF₃.

In some embodiments, R² and R⁴ are independently chlorine or CF₃.

In some embodiments, R¹ and R³ are hydrogen, X is nitrogen, and R⁵ is absent.

In some embodiments, R¹ and R³ are hydrogen.

In some embodiments, X is nitrogen and R⁵ is absent.

In some embodiments, X is carbon and R⁵ is hydrogen.

In some embodiments, the compound is

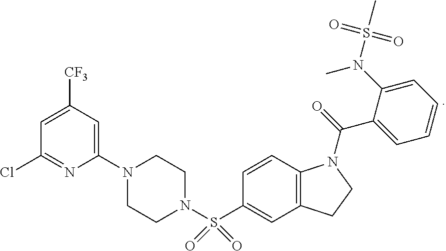 [SMILES: CN(c1ccccc1C(=O)N1CCc2cc(S(=O)(=O)N3CCN(c4cc(C(F)(F)F)cc(Cl)n4)CC3)ccc21)S(C)(=O)=O]

The present disclosure is directed to a compound of formula (B). or a pharmaceutically acceptable salt thereof:

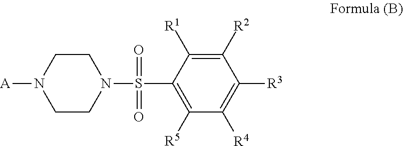

wherein
A is C₁-C₄ alkyl, aryl, substituted aryl, C₂-C₄ alkenyl, substituted C₅-C⁶ heteroaryl, cycloheterodialkenyl, or cyclohexyl;
R¹, R², and R⁵ are each independently hydrogen, halogen, or —COOH;
R³ is hydrogen or substituted amide;
R⁴ is hydrogen, halogen, or —COOH;
or R³ and R⁴ are interconnected to form an indoline, indole, pyrrolidine, or pyrrole; wherein the indoline, indole, pyrrolidine, or pyrrole is optionally substituted with —C(O)R⁶ or —S(O)(O)R⁶;
wherein the substituted amide is substituted with hydroxyl, halogen, C1-C6 alkyl, —NHC(O)R⁶, —NHC(O)C(O)NHOH, —NHC(O)NHR⁶, —NHR⁶C(O)NHOH, or —C(O)OR⁶ groups; and
wherein R⁶ is C₁-C₆ alkyl, substituted C₁-C₆ alkyl, C₁-C₆ alkenyl, C₁-C₆ substituted alkenyl, aryl, substituted aryl, heterocyclyl, hydrogen, halogen, hydroxyl, cyano, substituted sugar, or substituted heterocyclyl.

In some embodiments, the substituted aryl may be substituted with one or more of halogen, hydroxyl, carboxylic acid, C₁-C₂ alkyl substituted carboxylic acid, cycloheteroalkenyl, aryl, C₁-C₃ alkyl, or C₁ alkyl substituted with a halogen.

In some embodiments, the C₁ alkyl substituted with a halogen may be —CF₃.

In some embodiments, the substituted C₅-C₆ heteroaryl may be substituted with one or more of —CF₃ or Cl.

In some embodiments, R¹ may be Cl, F, or hydrogen.

In some embodiments, R² may be hydrogen.

In some embodiments, R⁵ may be hydrogen or carboxylic acid.

In some embodiments, R³ and R⁴ may be interconnected to form an indoline or indole.

The present disclosure is also directed to a compound of formulae (IIa) or (IIb), or a pharmaceutically acceptable salt thereof:

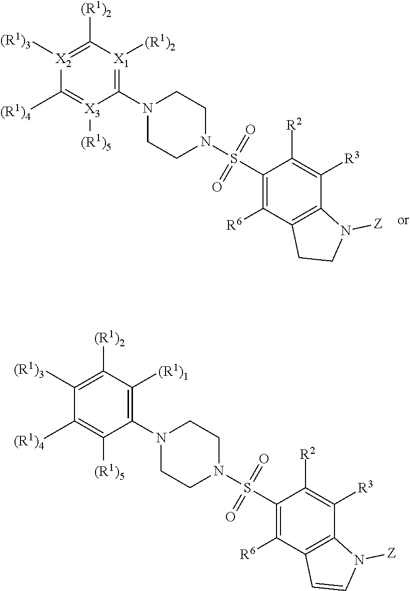

wherein
X₁, X₃, and X₅ are independently carbon or nitrogen;
(R¹)₁-(R¹)₅ are independently hydrogen, halogen, C₁-C₄ alkyl, C₁-C₆ substituted alkyl, aryl, C₁-C₆ alkenyl, hydroxyl, substituted heterocyclyl, carboxylic acid, substituted carboxylic acid, or absent;
R², R³ and R⁶ are each independently hydrogen, halogen, or —COOH;
Z is —C(O)—R⁷ or —S(O)(O)R⁷; and
   wherein R⁷ is methyl, amine, substituted amine, C₁-C₆ substituted alkyl, C₁-C₆ alkenyl, C₁-C₆ substituted alkenyl, aryl, substituted aryl, heterocyclyl, or substituted heterocyclyl.

In some embodiments, the C₁-C₆ substituted alkyl may be substituted with —CF₃, carboxylic acid, methyl substituted carboxylic acid, heterocycl, amine, methyl substituted amine, substituted ether, hydroxyamide, or methanesulfonamide.

In some embodiments, the substituted amine may be substituted with C₁-C₆ alkyl hydroxyamide, C₁-C₆ alkyl carboxylic acid, C₁-C₆ alkyl carboxylic acid, C₁-C₆ amine, or substituted C₁-C₆ amine.

The halogen may be F, Cl, or Br.

In some embodiments of formula (IIa),
(R¹)₁ and (R¹)₃ are hydrogen;
(R¹)₂ is —CF₃
(R¹)₄ is chlorine;
(R¹)₅ is absent;
X₁-X₂ are carbon;
X₃ is nitrogen;
R², R³, and R⁶ are hydrogen; and
Z is —C(O)R⁷;
R⁷ is substituted aryl;
wherein the substituted aryl is substituted with —NS(O)(O)CH₃.

In some embodiments of formula (IIb),
(R¹)₁, (R¹)₃, and (R¹)₅ are hydrogen;
(R¹)₂ is chlorine;
(R¹)₄ is —CF₃;
X₁-X₅ are carbon;
R², R³, R⁵, and R⁶ are hydrogen;
Z is —C(O)R⁷; and
R⁷ is substituted amine with formula —NHC₄H₈C(O)NHOH.

The present disclosure is also directed to a compound of formula (IIc), or a pharmaceutically acceptable salt thereof:

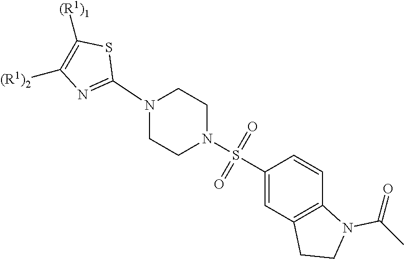

wherein
(R¹)₁ and (R¹)₂ may each independently be hydrogen or —CF₃.

The present disclosure is also directed to a compound of formulae (lid) or (lie), or a pharmaceutically acceptable salt thereof:

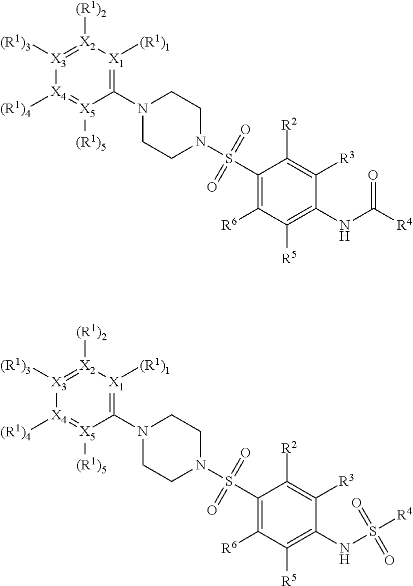

wherein
X₁-X₅ are independently carbon or nitrogen;
(R¹)₁-(R¹)₅ are independently hydrogen, halogen, C₁-C₆ alkyl, C₁-C₆ substituted alkyl, or absent;
R², R³, R⁵, and R⁶ are hydrogen;
R⁴ is C₁-C₆ alkyl, C₂-C₆ alkenyl, aryl, substituted aryl, —C(O)NHOH, —R⁷C(O)NHOH, —R⁷B(OH)₂, —R⁷, —NHR⁷, —NHR⁷C(O)OH, —NHR⁷C(O)NHOH, —NHR⁷C(O)OHC(O)OH, NHR⁷R⁸, —R⁷R⁸, or —C(O)R⁷,
   wherein R⁷ is C₁-C₆ alkyl or C₂-C₆ alkenyl; and
   wherein R⁸ is substituted sugar, heterocyclyl, substituted heterocyclyl, or substituted aryl.

In some embodiments, the C₁-C₆ substituted alkyl may be —CF₃.

In some embodiments, the halogen is F, Cl, or Br, or I.

In some embodiments of formula (IId),
(R¹)₁ and (R¹)₃ are hydrogen;
(R¹)₂ is —CF₃;
(R¹)₄ is chlorine;
(R¹)₅ is absent;
X₁-X₄ are carbon;
X₅ is nitrogen;
R², R³, R⁵, and R⁶ are hydrogen; and
R⁴ is —NHR⁷C(O)NHOH;
wherein R⁷ is pentyl.

In some embodiments of formula (lid),
(R¹)₁ and (R¹)₃ are hydrogen;
(R¹)₂ and (R¹)₄ are chlorine;
(R¹)₂ is absent;
X₁-X₅ are carbon;
R², R³, R⁵, and R⁶ are hydrogen; and
R⁴ is substituted aryl;
wherein the substituted aryl is substituted with methanesulfonamide.

In some embodiments of formula (lid),
(R¹)₁, (R¹)₃, and (R¹)₅ are hydrogen;
(R¹)₂ is chlorine;
(R¹)₄ is —CF₃;
X₁-X₅ are carbon;
X₅ is nitrogen;
R², R³, R⁵, and R⁶ are hydrogen; and
R⁴ is NHR⁷C(O)NHOH;
wherein R⁷ is pentyl.

In some embodiments the substituted sugar may be

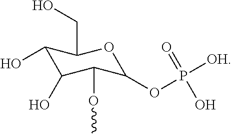

In some embodiments the substituted heterocyclyl is selected from the group consisting of

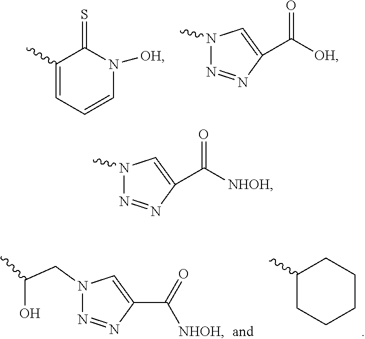

The present disclosure is futher directed to a compound of formulae (IIIa), (IIIb), (IVa), V), (VIa), or VIb) or a pharmaceutically acceptable salt thereof:

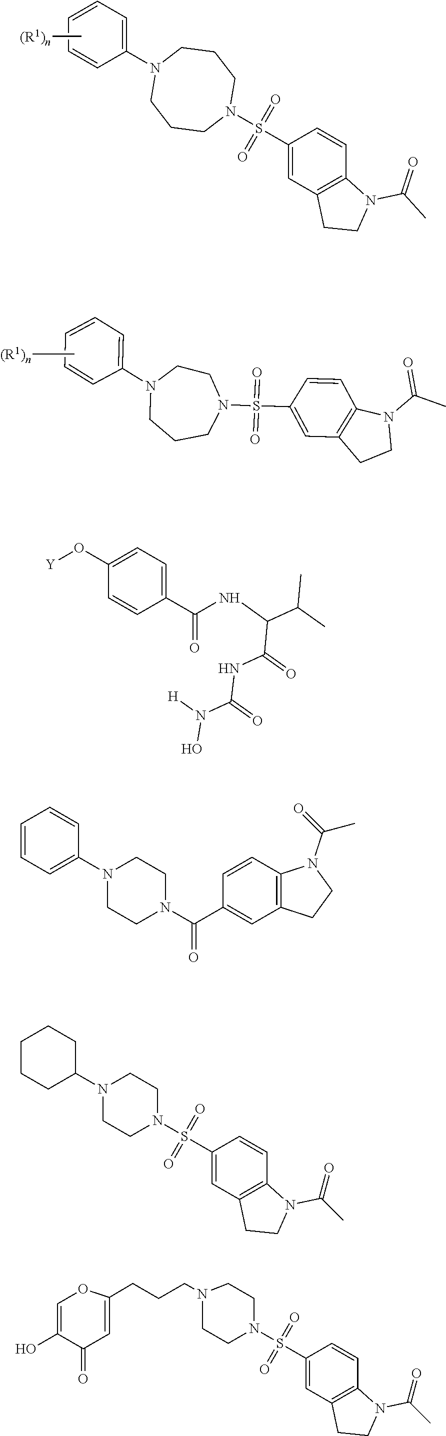

wherein
n is 1, 2, 3, 4, or 5;
(R¹)₁-(R¹)₅ are independently hydrogen, halogen, C₁-C₆ alkyl, or C₁-C₆ substituted alkyl; and
Y is C₁-C₁₀ alkyl.

The present disclosure is futher directed to a compound of formula (VII), or a pharmaceutically acceptable salt thereof:

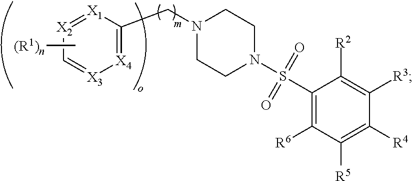

wherein
m is 0, 1, 2, 3, or 4;
n is 1, 2, 3, 4, or 5;
o is 0 or 1;
X₁-X₄ are independently carbon or nitrogen;
R¹ is independently selected from the group consisting of hydrogen, halogen, C₁-C₆ alkyl, substituted C₁-C₆ alkyl, aryl, substituted aryl, carboxylic acid, substituted carboxylic acid, and hydroxyl or absent;
R², R³, and R⁶ are independently selected from the group consisting of hydrogen, halogen, C₁-C₆ alkyl, and carboxylic acid;
R⁴ is hydrogen, hydroxyl, halogen, C₁-C₆ alkyl, —NHC(O)R⁷, —NHC(O)C(O)NHOH, ~NHC(O)NHR⁷; NHR⁷C(O)NHOH, or —C(O)OR⁷;
R⁵ is hydrogen, hydroxyl, halogen, C1-C6 alkyl, or —COOH;
or R⁴ and R⁵ are interconnected to form an indoline, indole, pyrrolidine, or pyrrole;
   wherein the indoline, indole, pyrrolidine, or pyrrole is optionally substituted with —C(O)R⁷ or —S(O)(O)R⁷; and
   wherein R⁷ is C₁-C₆ alkyl, substituted C₁-C₆ alkyl, C₁-C₆ alkenyl, C₁-C₆ substituted alkenyl, aryl, substituted aryl, heterocycyl, hydrogen, halogen, hydroxyl, cyano, substituted sugar, or substituted heterocycly.

In some embodiments, the compound or a pharmaceutically acceptable salt thereof of formula (V) may have a formula (VIIa):

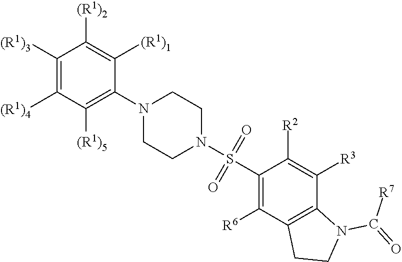

wherein
R¹ is independently selected from the group consisting of hydrogen, halogen, C₁-C₆ alkyl, substituted C₁-C₆ alkyl, aryl, substituted aryl, carboxylic acid, substituted carboxylic acid, and hydroxyl or absent;
R², R³, and R⁶ are independently selected from the group consisting of hydrogen, halogen, C₁-C₆ alkyl, and carboxylic acid;
R⁴ and R⁵ are interconnected to form the pyrrolidine substituted with —C(O)R⁷; and
wherein R⁷ is C₁-C₆ alkyl, substituted C₁-C₆ alkyl, C₁-C₆ alkenyl, C₁-C₆ substituted alkenyl, aryl, substituted aryl, heterocycyl, hydrogen, halogen, hydroxyl, cyano, substituted sugar, or substituted heterocycly.

The present disclosure is futher directed to a compound of formula (VIII), or a pharmaceutically acceptable salt thereof:

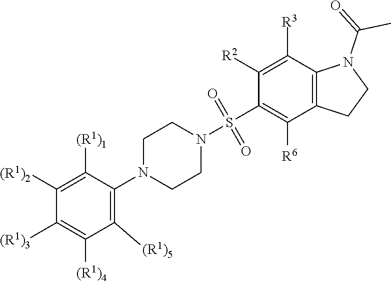

wherein
R¹ is independently selected from the group consisting of hydrogen, halogen, C₁-C₆ alkyl, substituted C₁-C₆ alkyl, aryl, substituted aryl, carboxylic acid, substituted carboxylic acid, and hydroxyl or absent; and
R², R³, and R⁶ are independently selected from the group consisting of hydrogen, halogen, C₁-C₆ alkyl, and carboxylic acid.

The present disclosure is futher directed to a compound of formula (IX), or a pharmaceutically acceptable salt thereof:

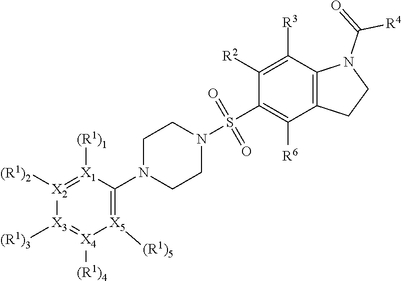

wherein
(R¹)₁-(R¹)₅ are each independently hydrogen, —CF₃, or absent;
X₁-X₅ are independently carbon or nitrogen;
R², R³, and R⁶ are hydrogen; and
R⁴ is C₁-C₆ alkyl, amino, or substituted amino.

The present disclosure is futher directed to a compound of formula (X), or a pharmaceutically acceptable salt thereof:

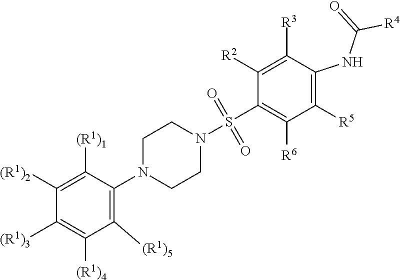

wherein
(R¹)₁-(R¹)₅ are each independently hydrogen or —CF₃;
R², R³, and R⁶ are each independently hydrogen, —CF₃, or —COOH;
R⁴ is —NHC(O)R⁷, —NHC(O)C(O)NHOH, hydrogen, —NHR⁷C(O)OH, —NHR⁷C(O)NOH, or —C(O)NHR⁷C(O)OHC(O)OH, —NHR⁷R⁸, —R⁷B(OH)₂, R⁷R⁸, —R⁸R⁷C(O)NHOH, or —R⁸R⁷;
wherein R⁷ is C₁-C₈ alkyl, C₁-C₆ alkenyl, substituted sugar, or substituted aryl; and
wherein R⁸ is substituted heterocyclyl.

The present disclosure is futher directed to a compound of formula (XI), or a pharmaceutically acceptable salt thereof:

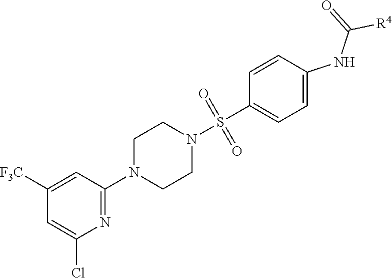

wherein
R⁴ is —NHR⁷C(O)NHOH or aryl substituted with methanesulfonamide;
R⁷ is C₁-C₈ alkyl;
wherein the methanesulfonamide is substituted with methyl or —R⁷C(O)NHOH; and the methansulfonamide is attached to the aryl ring through N or S.

The compounds of the present disclosure are listed in Table 1.

TABLE 1
Sulfonyl piperazine LpxH inhibitors.

| Compound ID | Structure |
|---|---|
| JH-LPH-01 (AZ1) | 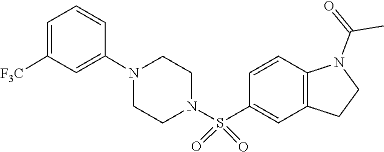 [SMILES: CC(=O)N1CCc2cc(S(=O)(=O)N3CCN(c4cccc(C(F)(F)F)c4)CC3)ccc21] |
| JH-LPH-04 | 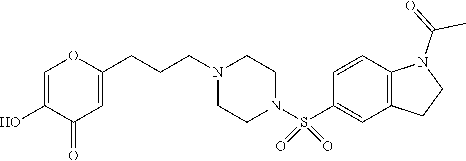 [SMILES: CC(=O)N1CCc2cc(S(=O)(=O)N3CCN(CCCc4cc(=O)c(O)co4)CC3)ccc21] |
| JH-LPH-05 | 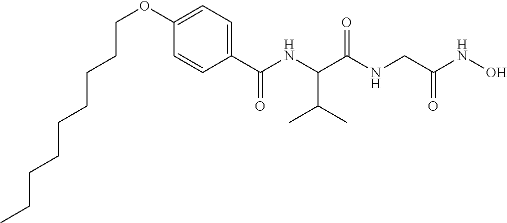 [SMILES: CCCCCCCCCOc1ccc(C(=O)NC(C(=O)NCC(=O)NO)C(C)C)cc1] |
| JH-LPH-06 | 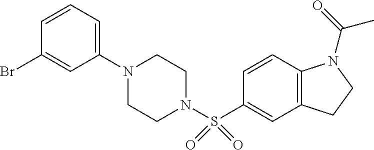 [SMILES: CC(=O)N1CCc2cc(S(=O)(=O)N3CCN(c4cccc(Br)c4)CC3)ccc21] |
| JH-LPH-07 | 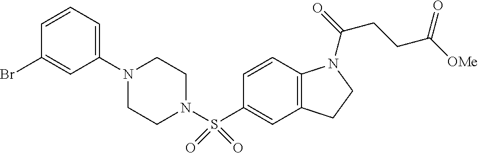 [SMILES: COC(=O)CCC(=O)N1CCc2cc(S(=O)(=O)N3CCN(c4cccc(Br)c4)CC3)ccc21] |
| JH-LPH-08 | 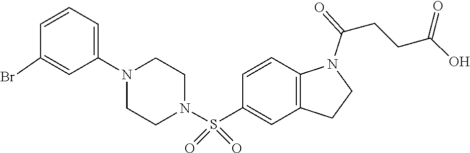 [SMILES: O=C(O)CCC(=O)N1CCc2cc(S(=O)(=O)N3CCN(c4cccc(Br)c4)CC3)ccc21] |
| JH-LPH-09 | 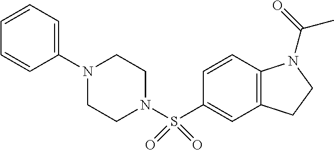 [SMILES: CC(=O)N1CCc2cc(S(=O)(=O)N3CCN(c4ccccc4)CC3)ccc21] |
| JH-LPH-10 | 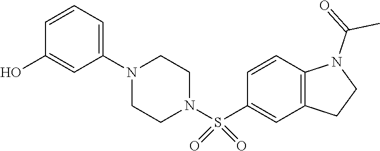 [SMILES: CC(=O)N1CCc2cc(S(=O)(=O)N3CCN(c4cccc(O)c4)CC3)ccc21] |
| JH-LPH-11 | 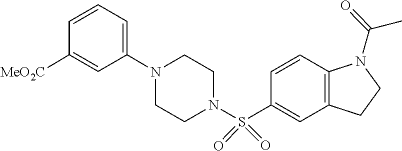 [SMILES: COC(=O)c1cccc(N2CCN(S(=O)(=O)c3ccc4c(c3)CCN4C(C)=O)CC2)c1] |
| JH-LPH-12 | 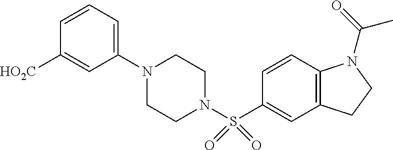 [SMILES: CC(=O)N1CCc2cc(S(=O)(=O)N3CCN(c4cccc(C(=O)O)c4)CC3)ccc21] |
| JH-LPH-13 | 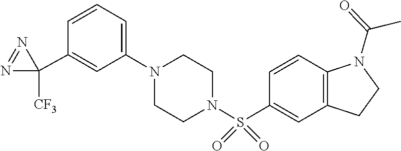 [SMILES: CC(=O)N1CCc2cc(S(=O)(=O)N3CCN(c4cccc(C5(C(F)(F)F)N=N5)c4)CC3)ccc21] |
| JH-LPH-14 | 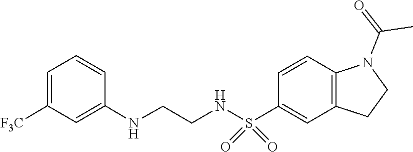 [SMILES: CC(=O)N1CCc2cc(S(=O)(=O)NCCNc3cccc(C(F)(F)F)c3)ccc21] |
| JH-LPH-15 | 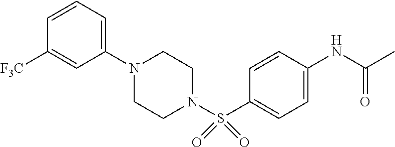 [SMILES: CC(=O)Nc1ccc(S(=O)(=O)N2CCN(c3cccc(C(F)(F)F)c3)CC2)cc1] |
| JH-LPH-17 | 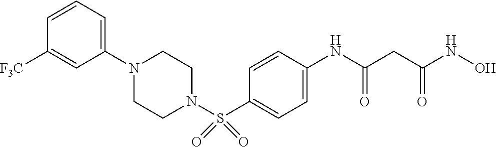 [SMILES: O=C(CC(=O)Nc1ccc(S(=O)(=O)N2CCN(c3cccc(C(F)(F)F)c3)CC2)cc1)NO] |
| JH-LPH-18 | 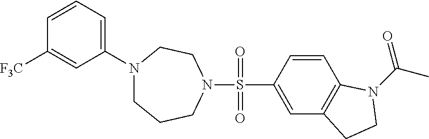 [SMILES: CC(=O)N1CCc2cc(S(=O)(=O)N3CCCN(c4cccc(C(F)(F)F)c4)CC3)ccc21] |
| JH-LPH-19 | 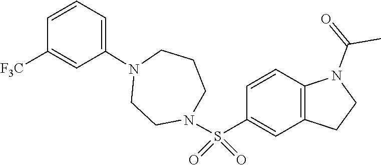 [SMILES: CC(=O)N1CCc2cc(S(=O)(=O)N3CCCN(c4cccc(C(F)(F)F)c4)CC3)ccc21] |
| JH-LPH-20 | 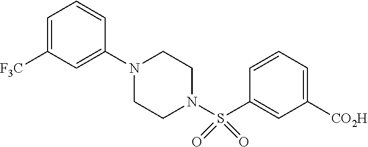 [SMILES: O=C(O)c1cccc(S(=O)(=O)N2CCN(c3cccc(C(F)(F)F)c3)CC2)c1] |
| JH-LPH-21 | 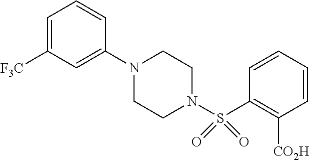 [SMILES: O=C(O)c1ccccc1S(=O)(=O)N1CCN(c2cccc(C(F)(F)F)c2)CC1] |
| JH-LPH-22 | 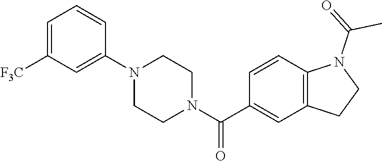 [SMILES: CC(=O)N1CCc2cc(C(=O)N3CCN(c4cccc(C(F)(F)F)c4)CC3)ccc21] |
| JH-LPH-23 | 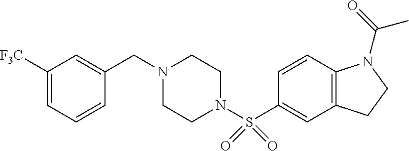 [SMILES: CC(=O)N1CCc2cc(S(=O)(=O)N3CCN(Cc4cccc(C(F)(F)F)c4)CC3)ccc21] |
| JH-LPH-24 | 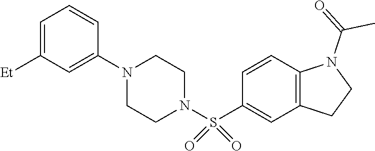 [SMILES: CCc1cccc(N2CCN(S(=O)(=O)c3ccc4c(c3)CCN4C(C)=O)CC2)c1] |
| JH-LPH-25 | 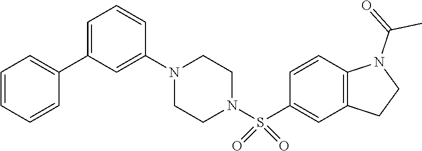 [SMILES: CC(=O)N1CCc2cc(S(=O)(=O)N3CCN(c4cccc(-c5ccccc5)c4)CC3)ccc21] |
| JH-LPH-26 | 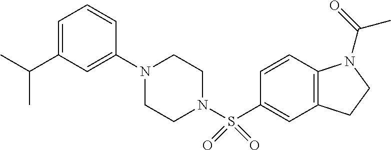 [SMILES: CC(=O)N1CCc2cc(S(=O)(=O)N3CCN(c4cccc(C(C)C)c4)CC3)ccc21] |
| JH-LPH-27 | 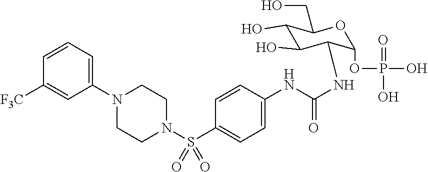 [SMILES: O=C(Nc1ccc(S(=O)(=O)N2CCN(c3cccc(C(F)(F)F)c3)CC2)cc1)NC1C(OP(=O)(O)O)OC(CO)C(O)C1O] |
| JH-LPH-28 | 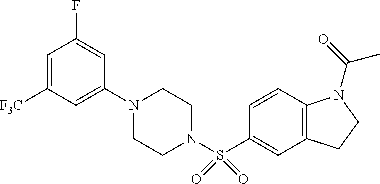 [SMILES: CC(=O)N1CCc2cc(S(=O)(=O)N3CCN(c4cc(F)cc(C(F)(F)F)c4)CC3)ccc21] |
| JH-LPH-33 | 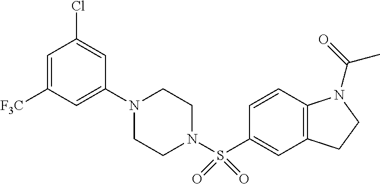 [SMILES: CC(=O)N1CCc2cc(S(=O)(=O)N3CCN(c4cc(Cl)cc(C(F)(F)F)c4)CC3)ccc21] |
| JH-LPH-36 | 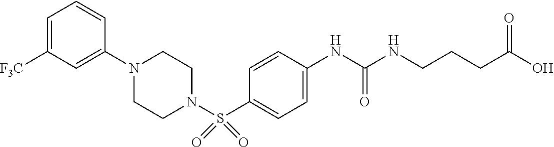 [SMILES: O=C(O)CCCNC(=O)Nc1ccc(S(=O)(=O)N2CCN(c3cccc(C(F)(F)F)c3)CC2)cc1] |
| JH-LPH-37 | 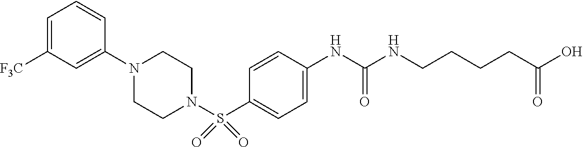 [SMILES: O=C(O)CCCCNC(=O)Nc1ccc(S(=O)(=O)N2CCN(c3cccc(C(F)(F)F)c3)CC2)cc1] |
| JH-LPH-38 | 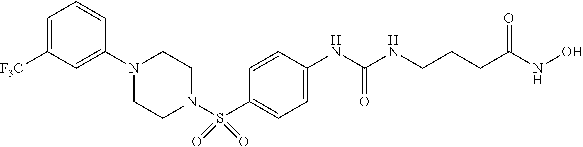 [SMILES: O=C(CCCNC(=O)Nc1ccc(S(=O)(=O)N2CCN(c3cccc(C(F)(F)F)c3)CC2)cc1)NO] |
| JH-LPH-39 | 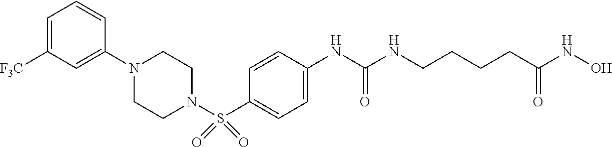 [SMILES: O=C(CCCCNC(=O)Nc1ccc(S(=O)(=O)N2CCN(c3cccc(C(F)(F)F)c3)CC2)cc1)NO] |
| JH-LPH-40 | 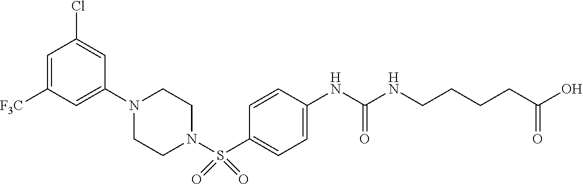 [SMILES: O=C(O)CCCCNC(=O)Nc1ccc(S(=O)(=O)N2CCN(c3cc(Cl)cc(C(F)(F)F)c3)CC2)cc1] |
| JH-LPH-41 | 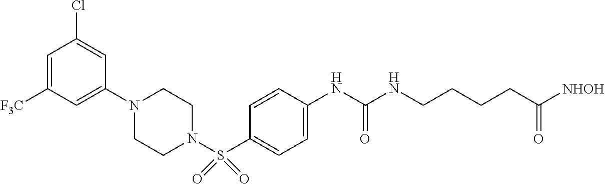 |
| JH-LPH-42 | 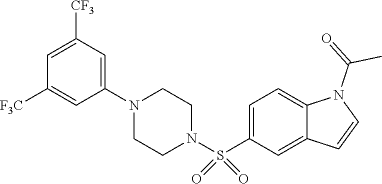 [SMILES: CC(=O)n1ccc2cc(S(=O)(=O)N3CCN(c4cc(C(F)(F)F)cc(C(F)(F)F)c4)CC3)ccc21] |
| JH-LPH-43 | 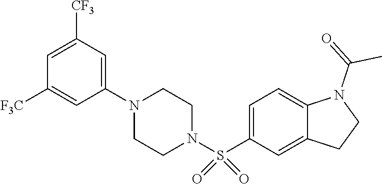 [SMILES: CC(=O)N1CCc2cc(S(=O)(=O)N3CCN(c4cc(C(F)(F)F)cc(C(F)(F)F)c4)CC3)ccc21] |
| JH-LPH-44 | 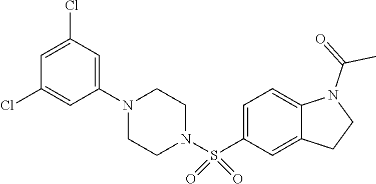 [SMILES: CC(=O)N1CCc2cc(S(=O)(=O)N3CCN(c4cc(Cl)cc(Cl)c4)CC3)ccc21] |
| JH-LPH-45 | 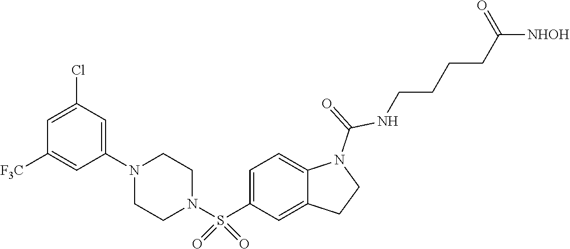 |
| JH-LPH-46 | 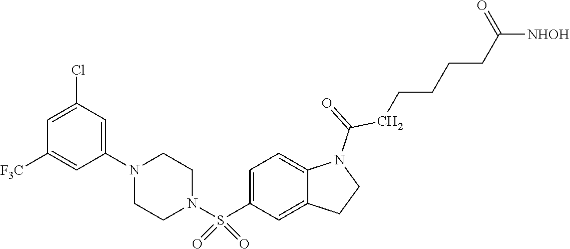 |
| JH-LPH-47 | 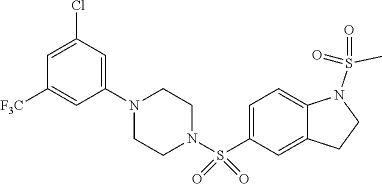 [SMILES: CS(=O)(=O)N1CCc2cc(S(=O)(=O)N3CCN(c4cc(Cl)cc(C(F)(F)F)c4)CC3)ccc21] |
| JH-LPH-48 | 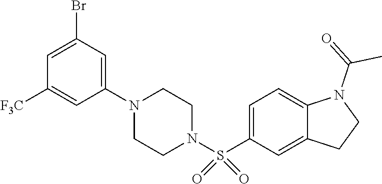 [SMILES: CC(=O)N1CCc2cc(S(=O)(=O)N3CCN(c4cc(Br)cc(C(F)(F)F)c4)CC3)ccc21] |
| JH-LPH-49 | 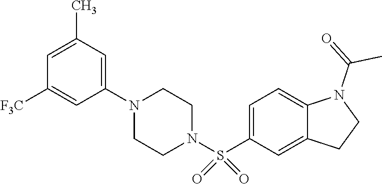 [SMILES: CC(=O)N1CCc2cc(S(=O)(=O)N3CCN(c4cc(C)cc(C(F)(F)F)c4)CC3)ccc21] |
| JH-LPH-50 | 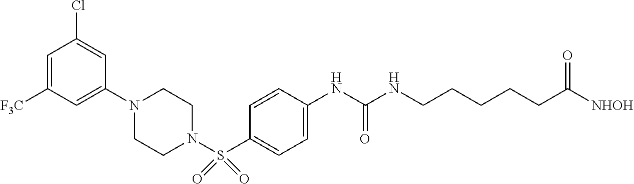 |
| JH-LPH-54 | 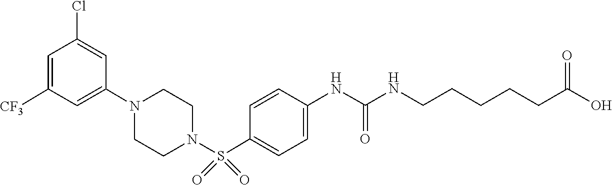 [SMILES: O=C(O)CCCCCNC(=O)Nc1ccc(S(=O)(=O)N2CCN(c3cc(Cl)cc(C(F)(F)F)c3)CC2)cc1] |
| JH-LPH-55 | 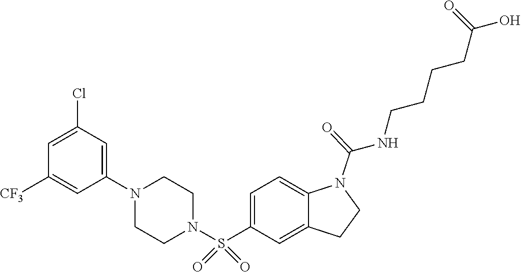 [SMILES: O=C(O)CCCCNC(=O)N1CCc2cc(S(=O)(=O)N3CCN(c4cc(Cl)cc(C(F)(F)F)c4)CC3)ccc21] |
| JH-LPH-56 | 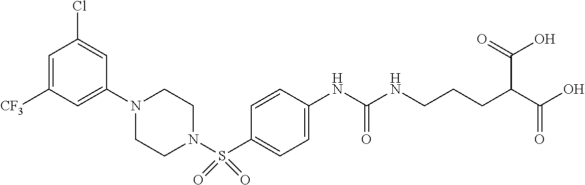 [SMILES: O=C(NCCCC(C(=O)O)C(=O)O)Nc1ccc(S(=O)(=O)N2CCN(c3cc(Cl)cc(C(F)(F)F)c3)CC2)cc1] |
| JH-LPH-59 | 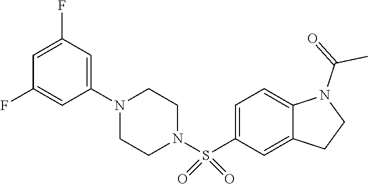 [SMILES: CC(=O)N1CCc2cc(S(=O)(=O)N3CCN(c4cc(F)cc(F)c4)CC3)ccc21] |
| JH-LPH-60 | 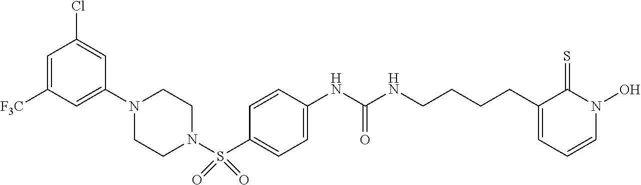 [SMILES: O=C(NCCCCc1cccn(O)c1=S)Nc1ccc(S(=O)(=O)N2CCN(c3cc(Cl)cc(C(F)(F)F)c3)CC2)cc1] |
| JH-LPH-61 | 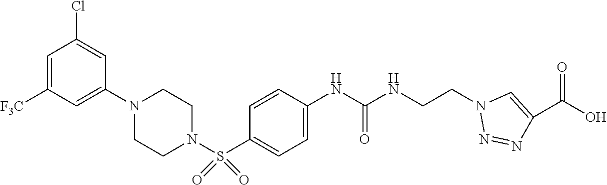 [SMILES: O=C(NCCn1cc(C(=O)O)nn1)Nc1ccc(S(=O)(=O)N2CCN(c3cc(Cl)cc(C(F)(F)F)c3)CC2)cc1] |
| JH-LPH-62 | 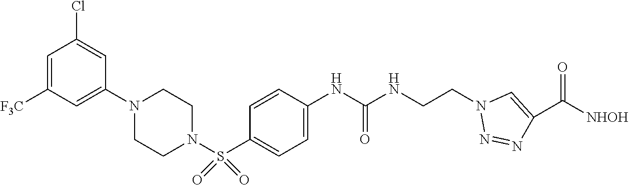 |
| JH-LPH-63 | 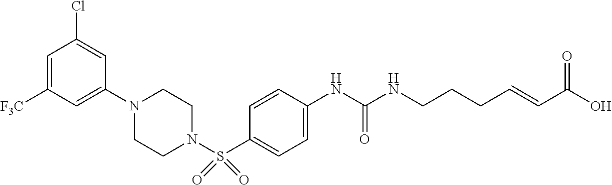 [SMILES: O=C(O)/C=C/CCCNC(=O)Nc1ccc(S(=O)(=O)N2CCN(c3cc(Cl)cc(C(F)(F)F)c3)CC2)cc1] |
| JH-LPH-64 | 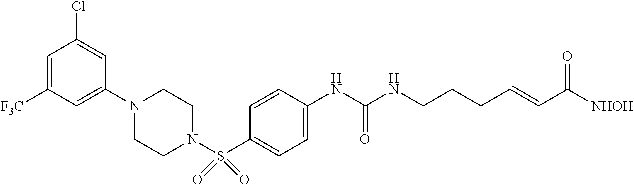 |
| JH-LPH-65 | 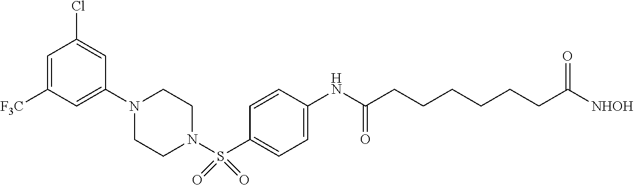 |
| JH-LPH-66 | 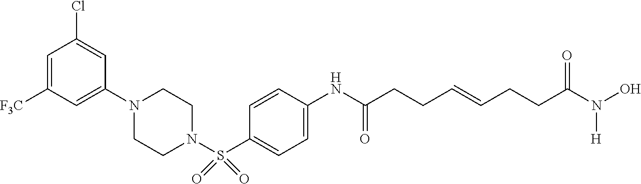 [SMILES: O=C(CC/C=C/CCC(=O)Nc1ccc(S(=O)(=O)N2CCN(c3cc(Cl)cc(C(F)(F)F)c3)CC2)cc1)NO] |
| JH-LPH-67 | 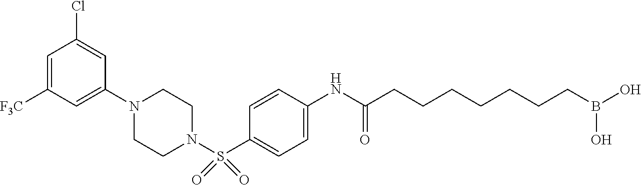 [SMILES: O=C(CCCCCCCB(O)O)Nc1ccc(S(=O)(=O)N2CCN(c3cc(Cl)cc(C(F)(F)F)c3)CC2)cc1] |
| JH-LPH-68 | 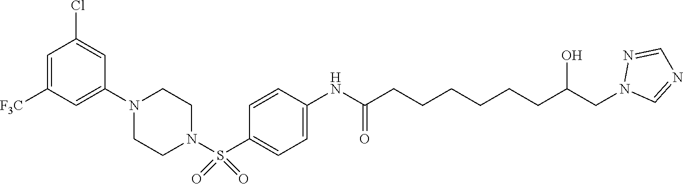 [SMILES: O=C(CCCCCCC(O)Cn1cncn1)Nc1ccc(S(=O)(=O)N2CCN(c3cc(Cl)cc(C(F)(F)F)c3)CC2)cc1] |
| JH-LPH-69 | 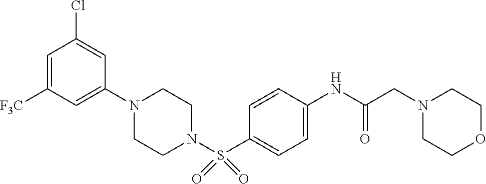 [SMILES: O=C(CN1CCOCC1)Nc1ccc(S(=O)(=O)N2CCN(c3cc(Cl)cc(C(F)(F)F)c3)CC2)cc1] |
| JH-LPH-70 | 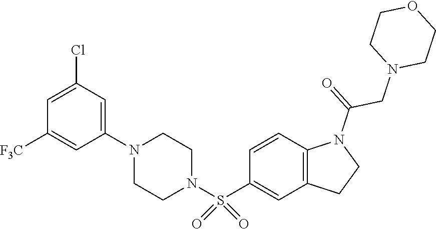 [SMILES: O=C(CN1CCOCC1)N1CCc2cc(S(=O)(=O)N3CCN(c4cc(Cl)cc(C(F)(F)F)c4)CC3)ccc21] |
| JH-LPH-71 | 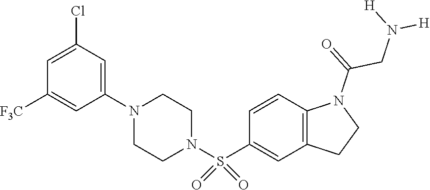 [SMILES: NCC(=O)N1CCc2cc(S(=O)(=O)N3CCN(c4cc(Cl)cc(C(F)(F)F)c4)CC3)ccc21] |
| JH-LPH-72 | 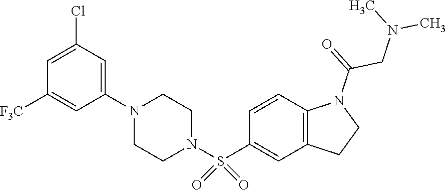 [SMILES: CN(C)CC(=O)N1CCc2cc(S(=O)(=O)N3CCN(c4cc(Cl)cc(C(F)(F)F)c4)CC3)ccc21] |
| JH-LPH-73 | 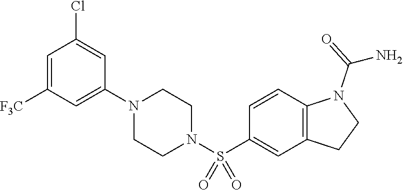 [SMILES: NC(=O)N1CCc2cc(S(=O)(=O)N3CCN(c4cc(Cl)cc(C(F)(F)F)c4)CC3)ccc21] |
| JH-LPH-74 | 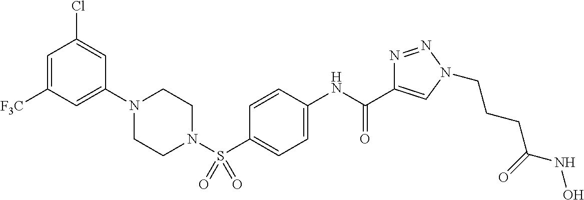 [SMILES: O=C(CCCn1cc(C(=O)Nc2ccc(S(=O)(=O)N3CCN(c4cc(Cl)cc(C(F)(F)F)c4)CC3)cc2)nn1)NO] |
| JH-LPH-75 | 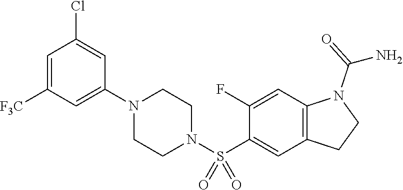 [SMILES: NC(=O)N1CCc2cc(S(=O)(=O)N3CCN(c4cc(Cl)cc(C(F)(F)F)c4)CC3)c(F)cc21] |
| JH-LPH-76 | 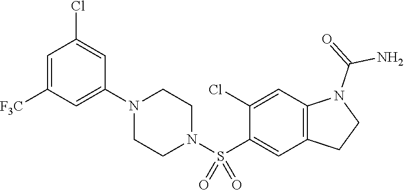 [SMILES: NC(=O)N1CCc2cc(S(=O)(=O)N3CCN(c4cc(Cl)cc(C(F)(F)F)c4)CC3)c(Cl)cc21] |
| JH-LPH-77 | 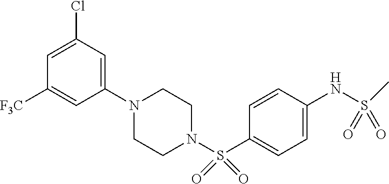 [SMILES: CS(=O)(=O)Nc1ccc(S(=O)(=O)N2CCN(c3cc(Cl)cc(C(F)(F)F)c3)CC2)cc1] |
| JH-LPH-78 | 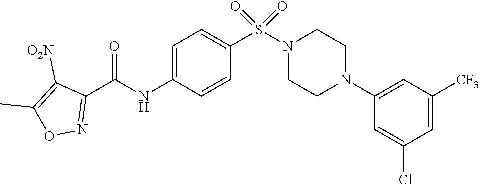 [SMILES: Cc1onc(C(=O)Nc2ccc(S(=O)(=O)N3CCN(c4cc(Cl)cc(C(F)(F)F)c4)CC3)cc2)c1[N+](=O)[O-]] |
| JH-LPH-79 | 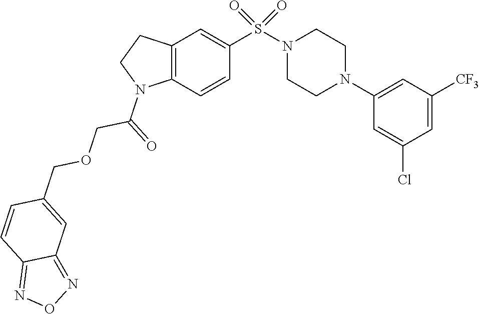 [SMILES: O=C(COCc1ccc2nonc2c1)N1CCc2cc(S(=O)(=O)N3CCN(c4cc(Cl)cc(C(F)(F)F)c4)CC3)ccc21] |
| JH-LPH-80 | 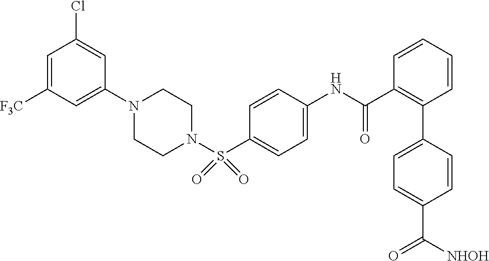 |
| JH-LPH-81 | 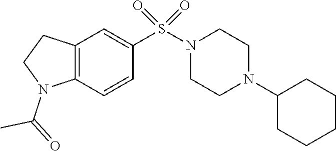 [SMILES: CC(=O)N1CCc2cc(S(=O)(=O)N3CCN(C4CCCCC4)CC3)ccc21] |
| JH-LPH-82 | 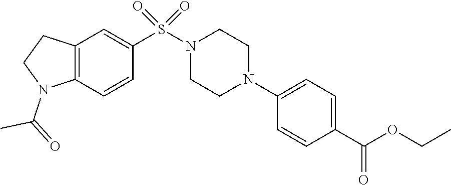 [SMILES: CCOC(=O)c1ccc(N2CCN(S(=O)(=O)c3ccc4c(c3)CCN4C(C)=O)CC2)cc1] |
| JH-LPH-83 | 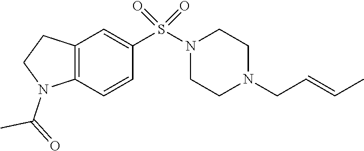 [SMILES: C/C=C/CN1CCN(S(=O)(=O)c2ccc3c(c2)CCN3C(C)=O)CC1] |
| JH-LPH-84 | 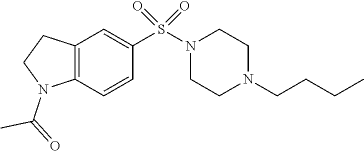 [SMILES: CCCCN1CCN(S(=O)(=O)c2ccc3c(c2)CCN3C(C)=O)CC1] |
| JH-LPH-85 | 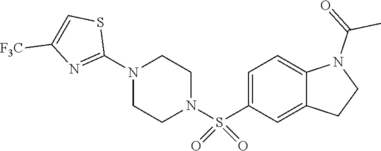 [SMILES: CC(=O)N1CCc2cc(S(=O)(=O)N3CCN(c4nc(C(F)(F)F)cs4)CC3)ccc21] |
| JH-LPH-86 | 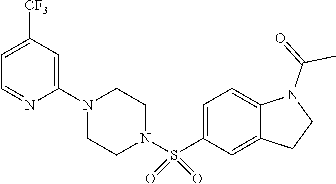 [SMILES: CC(=O)N1CCc2cc(S(=O)(=O)N3CCN(c4cc(C(F)(F)F)ccn4)CC3)ccc21] |
| JH-LPH-87 | 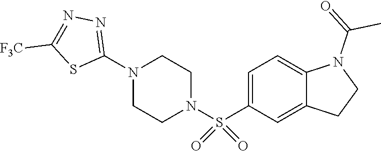 [SMILES: CC(=O)N1CCc2cc(S(=O)(=O)N3CCN(c4nnc(C(F)(F)F)s4)CC3)ccc21] |
| JH-LPH-88 | 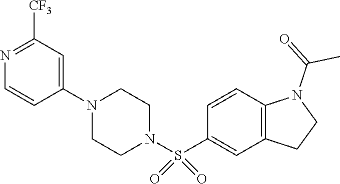 [SMILES: CC(=O)N1CCc2cc(S(=O)(=O)N3CCN(c4ccnc(C(F)(F)F)c4)CC3)ccc21] |
| JH-LPH-89 | 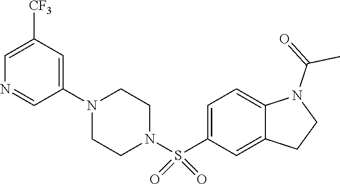 [SMILES: CC(=O)N1CCc2cc(S(=O)(=O)N3CCN(c4cncc(C(F)(F)F)c4)CC3)ccc21] |
| JH-LPH-90 | 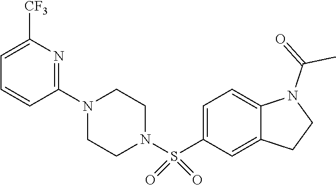 [SMILES: CC(=O)N1CCc2cc(S(=O)(=O)N3CCN(c4cccc(C(F)(F)F)n4)CC3)ccc21] |
| JH-LPH-91 | 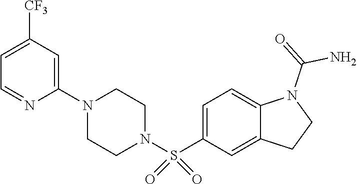 [SMILES: NC(=O)N1CCc2cc(S(=O)(=O)N3CCN(c4cc(C(F)(F)F)ccn4)CC3)ccc21] |
| JH-LPH-92 | 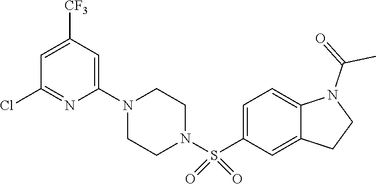 [SMILES: CC(=O)N1CCc2cc(S(=O)(=O)N3CCN(c4cc(C(F)(F)F)cc(Cl)n4)CC3)ccc21] |
| JH-LPH-93 | 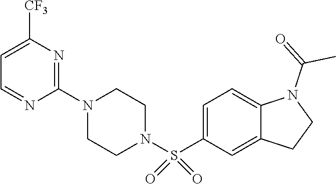 [SMILES: CC(=O)N1CCc2cc(S(=O)(=O)N3CCN(c4nccc(C(F)(F)F)n4)CC3)ccc21] |
| JH-LPH-94 | 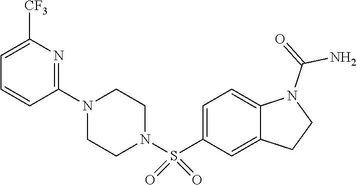 [SMILES: NC(=O)N1CCc2cc(S(=O)(=O)N3CCN(c4cccc(C(F)(F)F)n4)CC3)ccc21] |
| JH-LPH-95 | 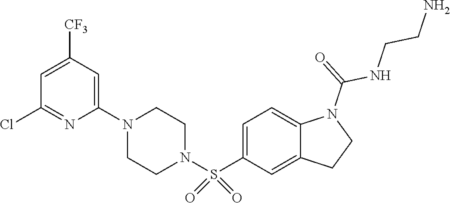 [SMILES: NCCNC(=O)N1CCc2cc(S(=O)(=O)N3CCN(c4cc(C(F)(F)F)cc(Cl)n4)CC3)ccc21] |
| JH-LPH-96 | 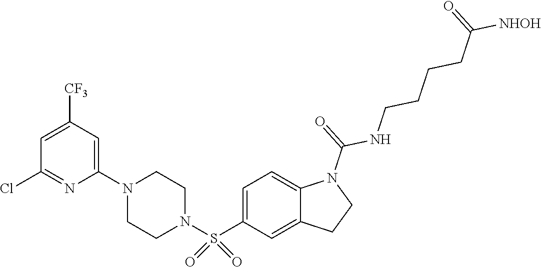 |
| JH-LPH-97 | 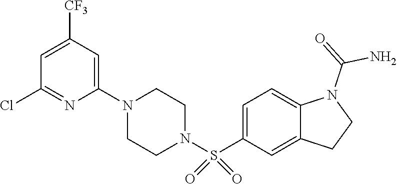 [SMILES: NC(=O)N1CCc2cc(S(=O)(=O)N3CCN(c4cc(C(F)(F)F)cc(Cl)n4)CC3)ccc21] |
| JH-LPH-98 | 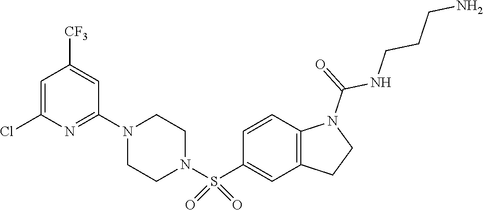 [SMILES: NCCCNC(=O)N1CCc2cc(S(=O)(=O)N3CCN(c4cc(C(F)(F)F)cc(Cl)n4)CC3)ccc21] |
| JH-LPH-99 | 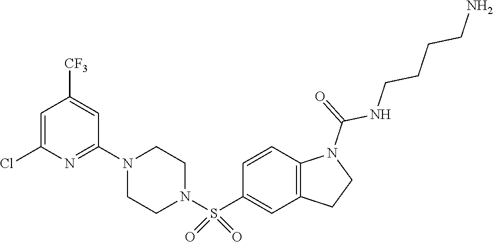 [SMILES: NCCCCNC(=O)N1CCc2cc(S(=O)(=O)N3CCN(c4cc(C(F)(F)F)cc(Cl)n4)CC3)ccc21] |
| JH-LPH-100 | 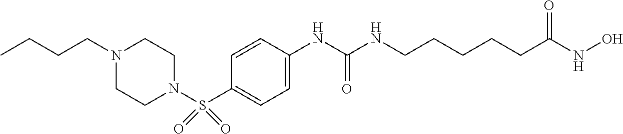 [SMILES: CCCCN1CCN(S(=O)(=O)c2ccc(NC(=O)NCCCCCC(=O)NO)cc2)CC1] |
| JH-LPH-101 | 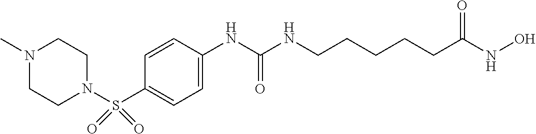 [SMILES: CN1CCN(S(=O)(=O)c2ccc(NC(=O)NCCCCCC(=O)NO)cc2)CC1] |
| JH-LPH-102 | 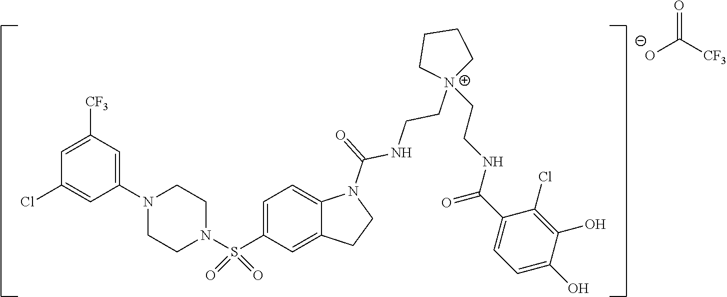 |
| JH-LPH-103 | 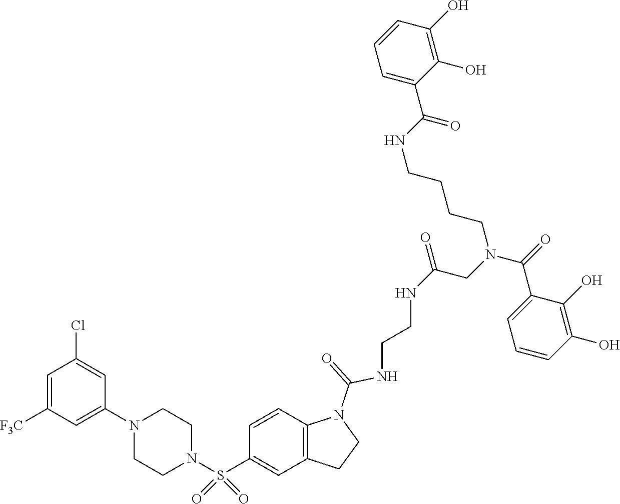 [SMILES: O=C(CN(CCCCNC(=O)c1cccc(O)c1O)C(=O)c1cccc(O)c1O)NCCNC(=O)N1CCc2cc(S(=O)(=O)N3CCN(c4cc(Cl)cc(C(F)(F)F)c4)CC3)ccc21] |
| JH-LPH-104 | 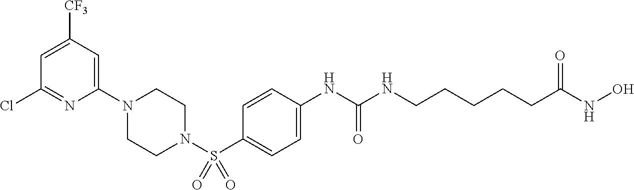 [SMILES: O=C(CCCCCNC(=O)Nc1ccc(S(=O)(=O)N2CCN(c3cc(C(F)(F)F)cc(Cl)n3)CC2)cc1)NO] |
| JH-LPH-105 | 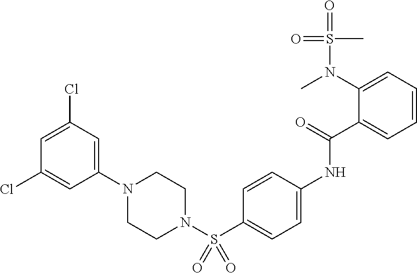 [SMILES: CN(c1ccccc1C(=O)Nc1ccc(S(=O)(=O)N2CCN(c3cc(Cl)cc(Cl)c3)CC2)cc1)S(C)(=O)=O] |
| JH-LPH-106 | 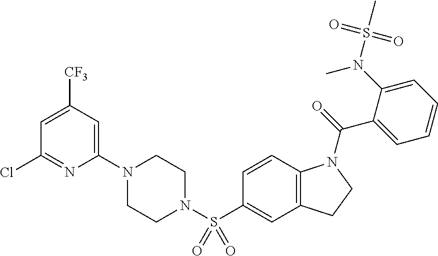 [SMILES: CN(c1ccccc1C(=O)N1CCc2cc(S(=O)(=O)N3CCN(c4cc(C(F)(F)F)cc(Cl)n4)CC3)ccc21)S(C)(=O)=O] |
| JH-LPH-107 | 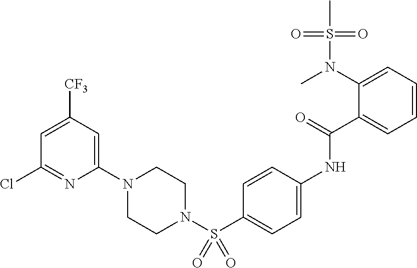 [SMILES: CN(c1ccccc1C(=O)Nc1ccc(S(=O)(=O)N2CCN(c3cc(C(F)(F)F)cc(Cl)n3)CC2)cc1)S(C)(=O)=O] |
| JH-LPH-108 series | 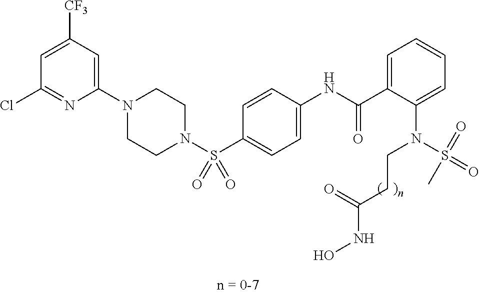 |
| JH-LPH-109 series | 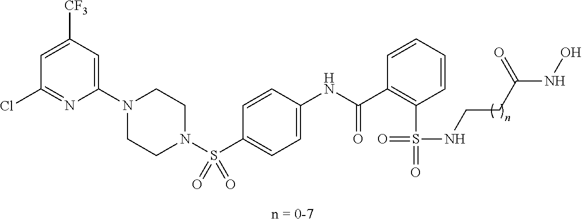 |

In one aspect, the present disclosure provides sulfonyl piperazine LpxH inhibitors with the potential of chelating the di-manganese cluster in the active site of LpxH.

In another aspect, the present disclosure provides di-manganese-chelating LpxH inhibitors with significantly enhanced potency in suppressing LpxH activity in vitro.

In one embodiment, the present disclosure provides a sulfonyl piperazine LpxH inhibitor comprising, consisting of, or consisting essentially of JH-LPH-45 or a variant, salt or ester thereof.

Another embodiment of the present disclosure provides a sulfonyl piperazine LpxH inhibitor comprising, consisting of, or consisting essentially of JH-LPH-50 or a variant, salt, or ester thereof.

In one embodiment, the present disclosure provides a sulfonyl piperazine LpxH inhibitor comprising, consisting of, or consisting essentially of JH-LPH-104 or a variant, salt or ester thereof.

In one embodiment, the present disclosure provides a sulfonyl piperazine LpxH inhibitor comprising, consisting of, or consisting essentially of JH-LPH-105 or a variant, salt or ester thereof.

In one embodiment, the present disclosure provides a sulfonyl piperazine LpxH inhibitor comprising, consisting of, or consisting essentially of JH-LPH-106 or a variant, salt or ester thereof.

In one embodiment, the present disclosure provides a sulfonyl piperazine LpxH inhibitor comprising, consisting of, or consisting essentially of JH-LPH-107 or a variant, salt or ester thereof.

### C. Pharmaceutical Compositions

In another aspect, the present disclosure provides compositions comprising one or more of sulfonyl piperazine LpxH inhibitors as described herein and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent a disease, such as a bacterial infection, the compounds described herein may be administered singly, as mixtures of one or more compounds or in mixture or combination with other agents (e.g., therapeutic agents) useful for treating such diseases and/or the symptoms associated with such diseases. Such agents may include, but are not limited to, antibiotics, NSAIDS, anti-inflammatory compounds, hemoperfusion devices, quorum sensing inhibitors, lytic bacteriophage, polyclonal or monoclonal antibodies, non-immune tolerizing approaches, liposome-based cytotoxin inhibitors, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plasticfoil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### D. Kits

Disclosed herein are cells transformed or transfected by one or more disclosed isolated nucleic acid molecules. Disclosed herein are cells transformed or transfected by an isolated nucleic acid molecule comprising a nucleic acid sequence encoding a disclosed chimeric fusion protein. Disclosed herein are cells transformed or transfected by an isolated nucleic acid molecule comprising a nucleic acid sequence encoding a disclosed chimeric fusion protein specific for phosphatidylserine (PS). Disclosed herein are cells transformed or transfected by an isolated nucleic acid molecule comprising a nucleic acid sequence encoding a chimeric fusion protein comprising a phosphatidylserine (PS) binding domain. Disclosed herein are cells transformed or transfected by an isolated nucleic acid molecule comprising a nucleic acid sequence encoding a chimeric fusion protein comprising a phosphatidylserine (PS) binding domain and an immunostimulatory domain. Disclosed herein are cells transformed or transfected by an isolated nucleic acid molecule comprising a nucleic acid sequence encoding a chimeric fusion protein comprising a phosphatidylserine (PS) binding domain operably linked an immunostimulatory domain. Disclosed herein are cells transformed or transfected by an isolated nucleic acid molecule comprising a nucleic acid sequence encoding a phosphatidylserine (PS) binding domain, a linker, and an immunostimulatory domain. Disclosed herein are cells transformed or transfected by an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule.

The present disclosure further provides kits comprising the compositions provided herein and for carrying out the subject methods as provided herein. For example, in one embodiment, a subject kit may comprise, consist of, or consist essentially of a sulfonylpiperazine LpxH inhibitor, or a salt, ester, thereof and/or pharmaceutical composition as provided herein.

In other embodiments, a kit may further include other components. Such components may be provided individually or in combinations, and may provide in any suitable container such as a vial, a bottle, or a tube. Examples of such components include, but are not limited to, one or more additional reagents, such as one or more dilution buffers; one or more reconstitution solutions; one or more wash buffers; one or more storage buffers, one or more control reagents and the like. Components (e.g., reagents) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### E. Methods

The sulfonyl piperazine LpxH inhibitors provided herein may be used to prevent and/or treat a disease, such as a bacterial infection, in a subject. Accordingly, another aspect of the present disclosure provides a method for preventing and/or treating a disease in a subject, the method comprising, consisting of, or consisting essentially of administering to the subject a therapeutically effective amount of a sulfonyl piperazine LpxH inhibitor as provided in Table 1 such that the disease is prevented and/or treated in the subject.

In one embodiment, the sulfonyl piperazine LpxH inhibitor comprises JH-LPH-45 or a salt, ester and/or pharmaceutical composition thereof.

In another embodiment, the sulfonyl piperazine LpxH inhibitor comprises JH-LPH-50 or a salt, ester and/or pharmaceutical composition thereof.

In another embodiment, the disease comprises a bacterial infection.

Another aspect of the present disclosure provides all that is described and illustrated herein.

### VII. EXAMPLES

The Examples that follow are illustrative of specific aspects of the invention, and various uses thereof. They set forth for explanatory purposes only and are not to be taken as limiting the invention.

### Example 1—Development of LpxH Inhibitors Chelating the Active Site Di-Manganese Metal Cluster of LpxH Results and Discussion

Design and synthesis of LpxH inhibitors: As metalloenzymes depend critically on their catalytic metal ion for activity, incorporation of a metal chelating group has been an effective strategy for enhancing the potency of metalloenzyme inhibitors. For example, the FDA-approved histone deacetylase (HDAC) inhibitor Vorinostat has a hydroxamic acid coordinating the catalytic Zn²⁺ in the HDAC active site as a monoanionic ligand through the carbonyl and hydroxyl donor atoms. Elvitegravir employs a quinolone carboxylic acid metal-binding pharmacophore to inhibit human immunodeficiency virus (HIV) integrase.

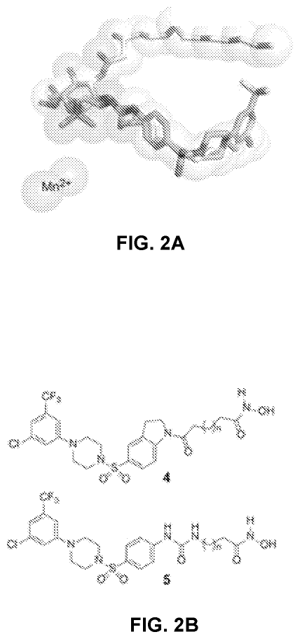

As LpxH harbors an active site di-manganese cluster that is required for structural integrity and catalysis, it was hypothesized that incorporation of a metal-binding group for the di-manganese cluster into LpxH inhibitors would similarly enhance their potency. It was noticed that the most recent LpxH inhibitor JH-LPH-41 (3), containing an aniline core with an N-acyl hydroxamate group extension, unexpectedly occupies the space of the hexose ring shared between the substrate and product as observed in the crystal structures of the lipid X-bound and JH-LPH-41-bound LpxH complexes (FIG. 2A; PDB IDs: 6PH9 and 6WII). Careful analysis of the KpLpxH/JH-LPH-41 structure showed that the N-hydroxy-5-ureido-pentanamide linker of JH-LPH-41 not only picks up additional hydrophobic interactions with Y125 of the insertion cap and 1171 on the loop connecting the cap back to the CLP core domain, but also forms two hydrogen bonds with the backbone amide and carbonyl group of M172 of the same loop as 1171. To achieve these interactions, the N-hydroxy-5-ureido-pentanamide linker must be slightly distorted and adopts a compact conformation. This observation led us to hypothesize that further structural modifications of the linker of JH-LPH-41 might prevent the linker from interacting with the insertion cap and the loop connecting the cap back to the CLP core domain and facilitate the chelation to the active site di-manganese cluster of LpxH. To test the hypothesis, the inventors embarked on chemical modifications of the hydroxamate linker of JH-LPH-41 (e.g., aniline vs indoline core, linker length and rigidity, etc) and assessed the effects of the modifications on the chelation of compounds to the di-manganese cluster of LpxH (FIG. 2B).

Indoline and aniline analogs: To develop a di-manganese-chelating LpxH inhibitor, evaluation began looking at the effect of acyl chain extension of JH-LPH-33 (2, FIG. 1B), which is the most potent LpxH inhibitor discovered thus far. In addition to the more favorable activity of the indoline core of AZ1 (1) over its corresponding aniline core (Scheme 1A), it was hypothesized that switching the aniline core of JH-LPH-41 (3) to the indoline core of AZ1 (1) might alter the orientation of the N-acyl linker of 3, thus directing the metal binding group, such as a hydroxamic acid, towards the active site di-manganese cluster.

To test this hypothesis, efforts were made on the synthesis of the indoline urea analogs 7 and 8 (Scheme 1A). Treatment of the known piperazinyl sulfonyl indoline 6 with triphosgene, coupling with ethyl 5-aminopentanoate, and basic hydrolysis afforded the corresponding carboxylic acid 7. Compound 7 was converted to the TBS-protected hydroxamic acid by treatment with ClCO₂Et/Et₃N/NH₂OTBS. Final TBS deprotection by TFA gave the indoline urea analog 8 in 78%. To investigate the effect of the urea linkage of 8 on the orientation of the N-acyl linker, we also prepared the corresponding amide analog 10. Acylation of 6 with ethyl 7-chloro-7-oxoheptanoate followed by hydrolysis proceeded smoothly to give carboxylic acid 9 (Scheme 1A). Treatment of 9 with ClCO₂Et/Et₃N/NH₂OTBS and final TBS deprotection by TFA completed the synthesis of the amide analog 10.

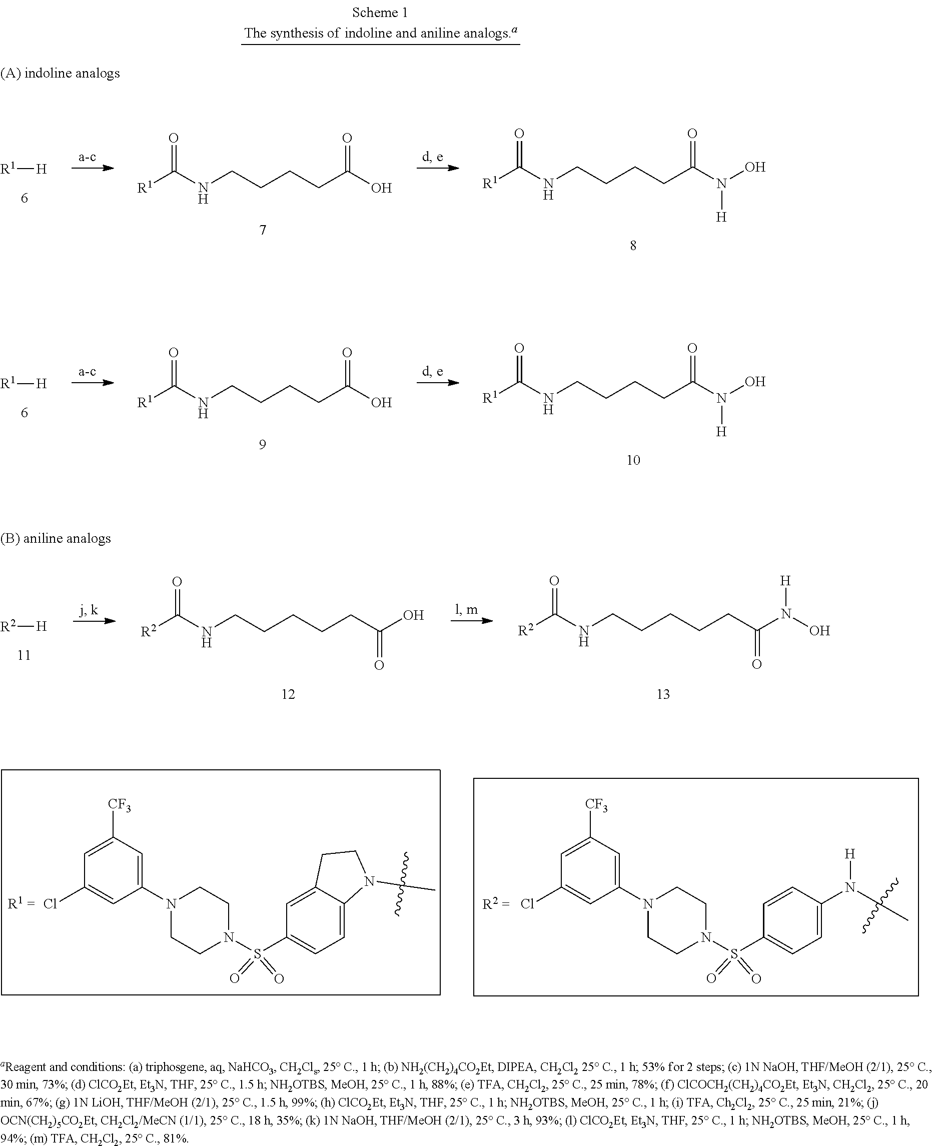

After the successful incorporation of a metal-binding pharmacophore for the di-manganese cluster into the indoline core of JH-LPH-33 (2), attention was turned to reexamining the design of aniline-based inhibitors such as JH-LPH-41 (3, FIG. 1B). It was reasoned that the N-acyl chain of 3 might not be sufficiently long enough to allow the terminal hydroxamate group to reach the di-manganese cluster in LpxH. To test this hypothesis, one-carbon homologated analogs 12 and 13 (Scheme 1B) were designed. Coupling of the known piperazinyl sulfonyl aniline 11 with ethyl 6-isocyanatohexanoate followed by hydrolysis under basic conditions afforded carboxylic acid 12. To install the hydroxamic acid moiety for metal binding, compound 12 was treated with ClCO₂Et/Et₃N/NH₂OTBS to give the corresponding TBS protected hydroxamic acid. Final TBS deprotection was accomplished by TFA to afford the desired one-carbon homologated analog 13 in 76% over two steps.

Analogs with a cyclic metal-binding group or a rigid linker: Antibiotics against Gram-negative bacteria need to find their way through the negatively charged LPS layer present on the surface of the outer membrane or translocate through porins to penetrate the hydrophobic outer membrane. Then, they need to cross the hydrophobic lipid bilayer of the inner membrane before they find their cytosolic targets such as LpxH. The flexible acyl chains of the above-mentioned compounds may restrict the membrane permeability of the compounds. To investigate the effect of structural rigidification of the flexible acyl chain on of 13 on activity, the acyclic hydroxamic acid moiety of 13 was replaced with a more rigid cyclic metal binding group. The 1-hydroxy-2(1H)-pyridinethione metal binding group was originally developed by Cohen and co-workers and has been used in matrix metalloproteinase inhibitors. CDI coupling of 11 to 14 (see the Supporting Information for details) completed the synthesis of the hydroxy pyridinethione analog 15 (Scheme 2).

The N-acyl linker of 13 was also made more rigid by incorporating a double bond or a triazole ring. For the synthesis of the (E)-olefinic linker analog 18, 11 was reacted with tert-butyl (E)-6-aminohex-2-enoate (16) (see Appendix A for details), CDI, and DIPEA to give the corresponding urea intermediate (Scheme 2). Treatment of the urea intermediate with TFA afforded carboxylic acid 17. Carboxylic acid 17 was converted to the final olefinic linker analog 18 following the same procedure used for 13. Another rigid analog that was designed was the triazole linker analog 20 (Scheme 2). To synthesize 20, the process started with coupling 11 with 1-chloro-2-isocyanatoethane (89%) followed by treating with NaN₃ and TBAI to give azide 19 (59%). The click chemistry of azide 19 and N-hydroxypropiolamide provided the desired triazole analog 20, albeit in low yield (28%).

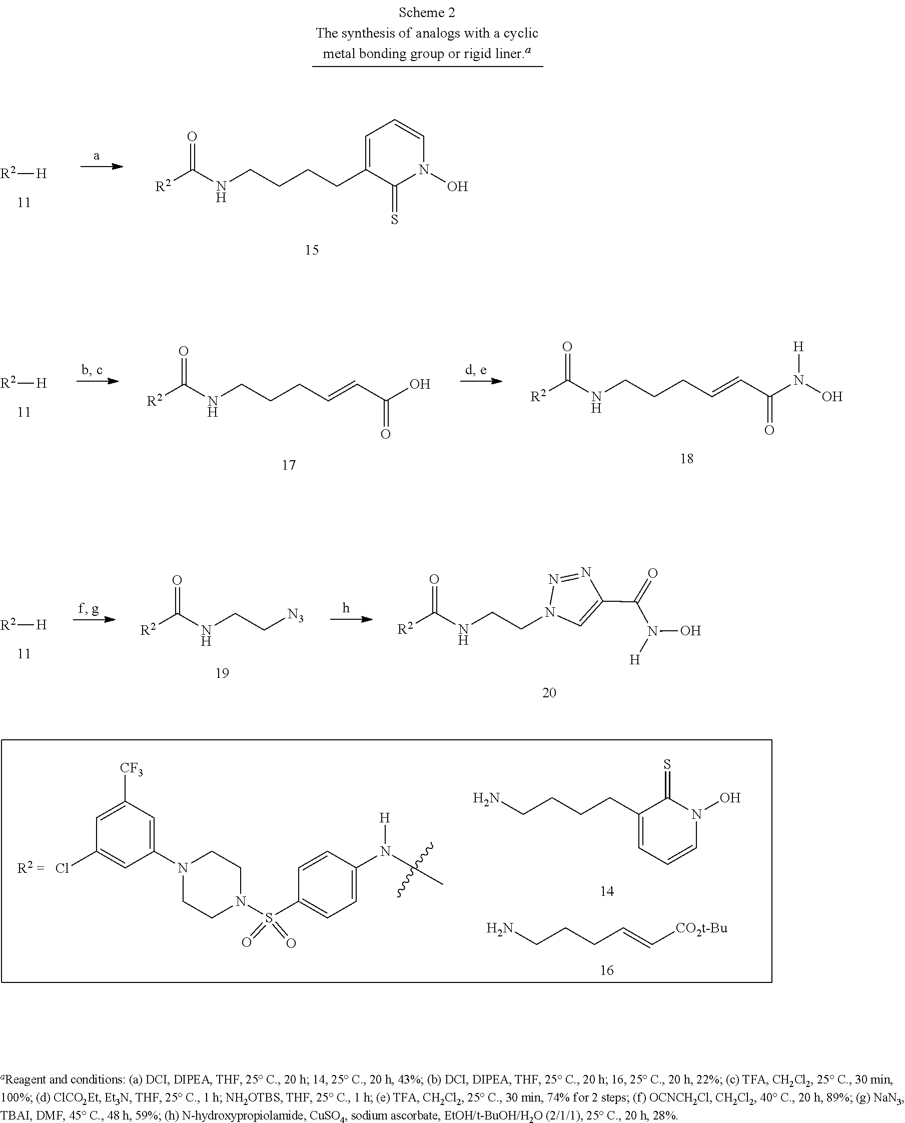

Evaluation of LpxH inhibition by analogs: After completing the synthesis of sulfonyl piperazine compounds with various forms of linkers, the KpLpxH inhibition was biochemically characterized by these analogs at 0.1 μM using the nonradioactive, colorimetric malachite green assay that we had previously reported. It was found that compound 8 was more potent in inhibiting KpLpxH (vᵢ/v₀=18±1%) than 2 (vᵢ/v₀=21%) (Table 1). Such an enhanced potency requires the presence of the hydroxamate group, as its substitution with a carboxylate group in compound 7 reduced the compound activity (vᵢ/v₀=30±6%). This result suggests that the superior inhibitory effect of 8 is likely derived from chelation of the di-manganese cluster.

It was also found that compound 13, with a one-carbon extension compared to the acyl hydroxamate group of 3, nearly completely inhibited KpLpxH at 0.1 μM, leaving 3±2% of the enzymatic activity (vᵢ/v₀), whereas KpLpxH retained 36% activity in the presence of 0.1 μM of 3 (Table 2). Compound 13 is also a more potent inhibitor than 2 at 0.1 μM (vᵢ/v₀=21%). Such a significant improvement in potency was lost when the hydroxamate group was substituted with the carboxylate group (12; vᵢ/v₀=57±3%), suggesting that the inhibitory effect similarly requires the hydroxamate group and its ability to chelate the di-manganese cluster.

Unfortunately, neither substituting the hydroxamate group with a cyclic metal binding group as in 15 (vᵢ/v₀=39±6% inhibition) nor introducing a double bond as in 18 (vᵢ/v₀=34±7% inhibition) or a cyclic ring as in 20 (vᵢ/v₀=59±7% inhibition) next to the hydroxamate group enhanced the compound activity when tested at 0.1 μM concentration (Table 2). It is likely due to the loss of the di-manganese chelation in these

TABLE 2
Percentage activity of KpLpxH in the presence of 0.1 μM LpxH inhibitors

| | | Percentage | Percentage |
|---|---|---|---|
| Compounds | Structure | Activity^{[a]} | Inhibition |
| 1 (AZI) | 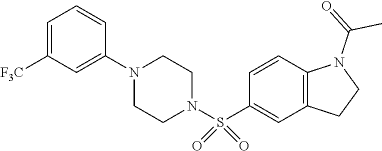 [SMILES: CC(=O)N1CCc2cc(S(=O)(=O)N3CCN(c4cccc(C(F)(F)F)c4)CC3)ccc21] | 78 | 22 |
| 2 (JH-LPH-33) | 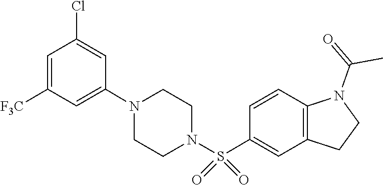 [SMILES: CC(=O)N1CCc2cc(S(=O)(=O)N3CCN(c4cc(Cl)cc(C(F)(F)F)c4)CC3)ccc21] | 21 | 79 |
| 3 (JH-LPH-41) | 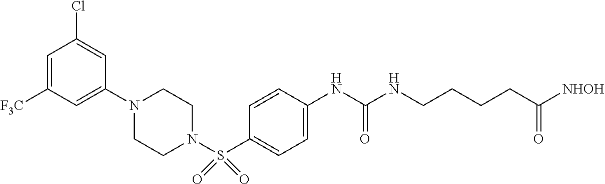 | 36 | 64 |
| 7 (JH-LPH-55) | 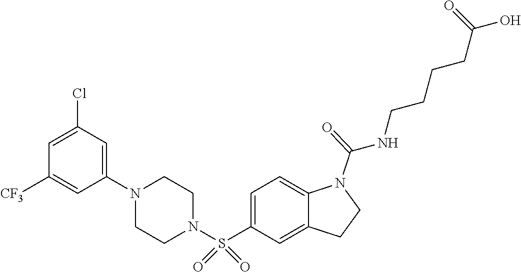 [SMILES: O=C(O)CCCCNC(=O)N1CCc2cc(S(=O)(=O)N3CCN(c4cc(Cl)cc(C(F)(F)F)c4)CC3)ccc21] | 30 ± 6 | 70 |
| 8 (JH-LPH-45) | 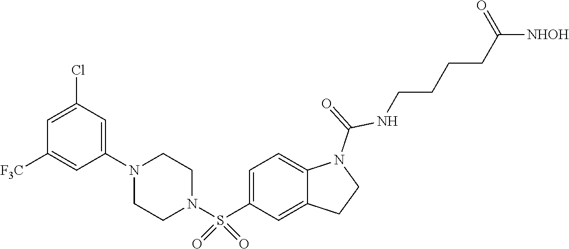 | 18 ± 1 | 82 |
| 10 (JH-LPH-46) | 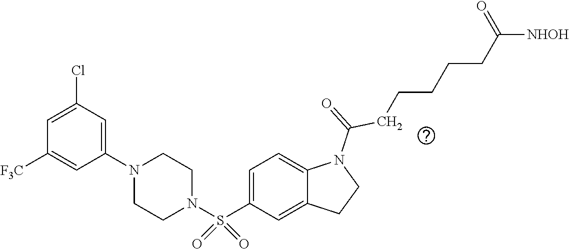 | 93 ± 7 | 7 |
| 12 (JH-LPH-54) | 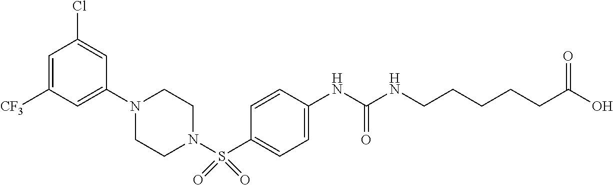 [SMILES: O=C(O)CCCCCNC(=O)Nc1ccc(S(=O)(=O)N2CCN(c3cc(Cl)cc(C(F)(F)F)c3)CC2)cc1] | 57 ± 3 | 43 |
| 13 (JH-LPH-50) | 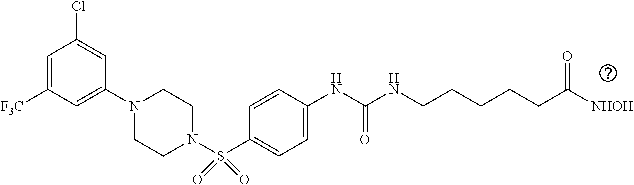 | 3 ± 2 | 97 |
| 15 (JH-LPH-60) | 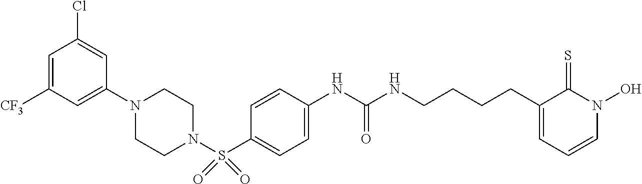 [SMILES: O=C(NCCCCc1cccn(O)c1=S)Nc1ccc(S(=O)(=O)N2CCN(c3cc(Cl)cc(C(F)(F)F)c3)CC2)cc1] | 39 ± 6 | 61 |
| 18 (JH-LPH-64) | 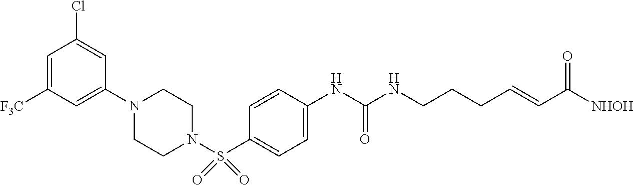 | 34 ± 7 | 66 |
| 20 (JH-LPH-62) | 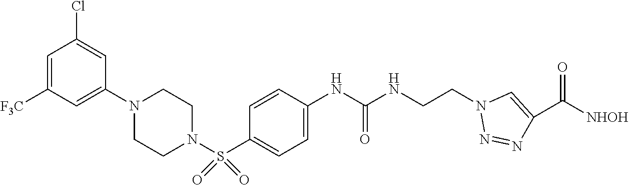 | 59 ± 7 | 41 |

^{[a]}Percentage activity was normalized against the specific activity of KpLpxH measured in the presence of 10% DMSO (309 ± 12 μmol/min/mg). Errors represent the standard percent error.

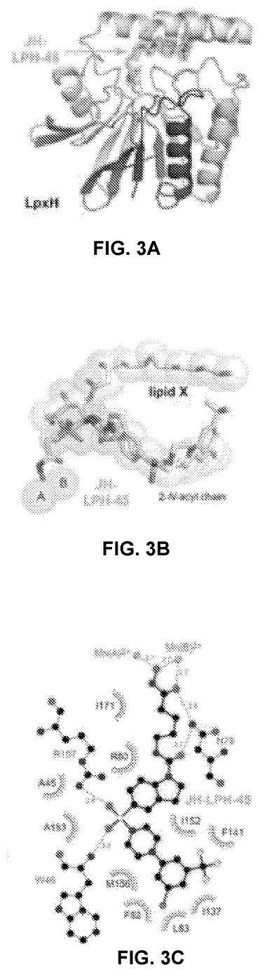

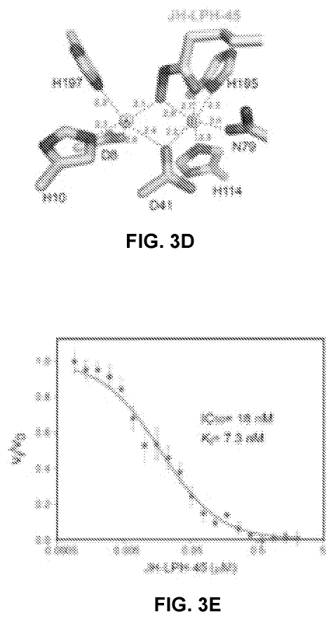

Structures of K. pneumoniae LpxH in complex with 8 (JH-LPH-45) and 13 (JH-LPH-50): In order to gain a better understanding of the nature of the interaction of 8 (JH-LPH-45) with K. pneumoniae LpxH (KpLpxH), the co-crystal structure of the KpLpxH/8 complex at 1.74 Å (FIG. 3) was determined. The location of 8 is very well defined by the omit map density. Consistent with the design and similar to previously reported LpxH inhibitors, the crystal structure of KpLpxH/8 showed that the 5-((4-(3-chloro-5-(trifluoromethyl)phenyl)piperazin-1-yl)sulfonyl)indoline moiety of 8 occupies the 2-N-acyl chain binding chamber of LpxH defined by a cluster of hydrophobic residues, such as A45, F82, L83, 1137, F141, 1152, M156, A153, 1171 (FIGS. 3A-C). The sidechain of R80 forms a prototypical cation-π interaction with the indoline group. Additionally, the sulfonyl oxygen atoms form hydrogen bonds with sidechain of R157 and backbone of W46, and the urea group forms a hydrogen bond with the sidechain of N79 (FIG. 3C). However, compound 8 is distinct from previously reported LpxH inhibitors in that it chelates the di-manganese cluster in the active site of LpxH. It does so with its acyl chain extending towards the di-manganese cluster with the N-hydroxyl group bridging two manganese ions A and B and the carbonyl oxygen of the hydroxamic acid interacting with manganese ion B (FIG. 3D). As a result, manganese ion A is penta-coordinated in a pseudo-square pyramidal configuration with the hydroxamate N-hydroxyl group of 6 occupying a base vertex, whereas manganese ion B is hexa-coordinated in a pseudo-octahedral geometry, with the N-hydroxyl group and carbonyl oxygen atom of the hydroxamic acid group of 6 occupying two vertices. Accordingly, compound 8 displayed a significant reduction of the IC₅₀ value (IC₅₀=18 nM, Kᵢ=7.3 nM, FIG. 3E) in comparison with the reported value for 2 (IC₅₀=26 nM, Kᵢ=10 nM) which was the most potent LpxH inhibitor discovered thus far. Intriguingly, replacing the urea group with the corresponding amide group (compound 10) reduced the compound activity (vᵢ/v₀=93±7%) despite a lack of interaction of the urea nitrogen atoms with the enzyme, suggesting that the geometric constraints provided by the urea linker are beneficial for activity.

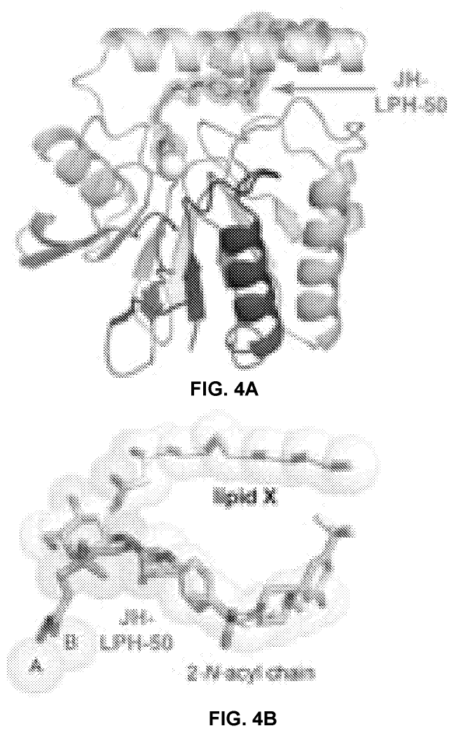

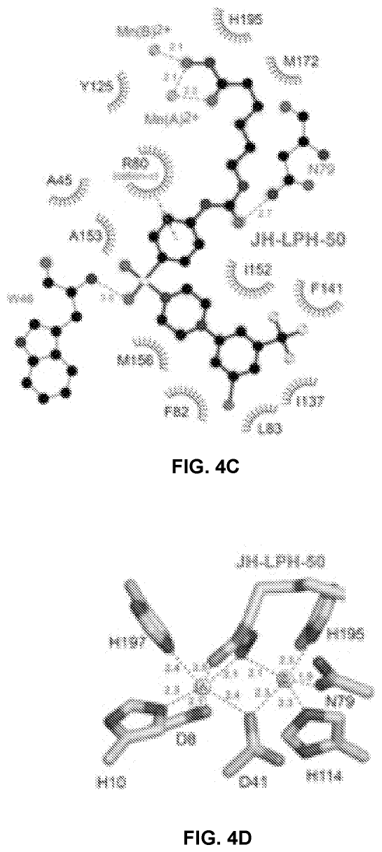

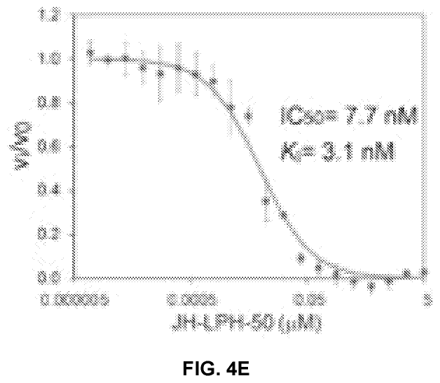

To verify that the gain of activity of 13 (JH-LPH-50) was due to chelation of the di-manganese cluster, the crystal structure of the KpLpxH/13 complex resolved at 1.73 Å resolution was also analyzed (FIG. 4A). The density for compound 13 is also very well defined. Similar to 8 (FIG. 3), compound 13 also has its sulfonyl piperazine tail group occupying the 2-N-acyl chain chamber of LpxH and its acyl hydroxamate reaching into the active site and chelating the di-manganese cluster (FIGS. 4A-C). However, there is a major distinction between 8 and 13: although the N-hydroxyl group of 13 similarly bridges both manganese ions, its carbonyl oxygen coordinates manganese ion A instead of B (FIGS. 4 C &D). As a result, manganese ion A is now hexa-coordinated in a pseudo-octahedral geometry, whereas manganese ion B is penta-coordinated in a pseudo square pyramidal geometry. The distance from the carbonyl oxygen of the hydroxamate group is closer for manganese ion A to 13 (2.5 A) than that of manganese ion B to 8 (2.7 A), suggesting that it is energetically more favorable. Such an effect may contribute to the lower IC₅₀ value of 13 (IC₅₀=7.7 nM, Kᵢ=3.1 nM; FIG. 4E) than that of 8 (IC₅₀=18 nM, Kᵢ=7.3 nM; FIG. 3E).

### Evaluation of Antibiotic Activity of Compounds 8 (JH-LPH-45) and 13 (JH-LPH-50):

To evaluate the effect of di-manganese chelation on antibiotic activity, compounds 8 and 13 were tested against K. pneumoniae (ATCC 10031) in vitro (Table 3). Despite the significant reduction of Kᵢ values of 8 (7.3 nM) and 13 (3.1 nM) in comparison with 2 (10 nM) in enzymatic assays, compounds 8 and 13 displayed lower antibiotic activity (MIC=18.7 μg/mL for 8 and MIC=3.3 μg/mL for 13) than the non-metal chelating compound 2 (MIC=0.83 μg/mL). Such a discrepancy was presumably caused by the flexible acyl chain that hindered the membrane permeability of the compounds as suggested by the eNTRY rule.

TABLE 3
Antibiotic activity of LpxH inhibitors

| | 1 | 2 | 8 | 13 |
|---|---|---|---|---|
| Compounds | (AZI) | (JH-LPH-33) | (JH-LPH-45) | (JH-LPH-50) |
| MIC (μg/mL) | >64 | 0.83 | 18.7 | 3.3 |

Both JH-LPH-45 and JH-LPH-50 leverage a terminal hydroxamate group to chelate the di-manganese cluster and enhance ligand-LpxH interaction. The hydroxamate group has been used as a metal chelating group in FDA approved histone deacetylase inhibitors, such as Belinostat, Panobinostat, and Vorinostat. Although hydroxamate-containing LpxH inhibitors may inhibitor human metalloenzymes, the unique lipid binding feature of LpxH could be leveraged to ensure the specificity of LpxH inhibitor and minimize off-target effects. Furthermore, replacing the hydroxamate group with alternative metal-chelating groups may further enhance the selectivity of LpxH inhibitors containing a metal chelating group, thus mitigating the potential off-target consequence.

### CONCLUSION

The LpxH enzyme in the lipid A biosynthetic pathway is a promising antibiotic target. Building on the sulfonyl piperazine scaffold of AZ1 (1), it was previously reported of more potent AZ1 analogs, such as JH-LPH-33 (2) with significant improvement of antibiotic activity and JH-LPH-41 (3) that reaches the polar binding pocket near the active site. After careful analyses of the structures of KpLpxH/JH-LPH-33 (2) and KpLpxH/JH-LPH-41 (3), the inventors designed JH-LPH-45 (8) and JH-LPH-50 (13) by switching the aniline core to the corresponding indoline core (8) or extending the linker by a methylene unit (13). Consistent with their prediction, both 8 and 13 chelate the di-manganese cluster in the active site of KpLpxH. Most notably, the chelation of the active site di-manganese cluster of KpLpxH significantly improves the potency of compounds 8 and 13, reducing the Kᵢ values from 10 nM (JH-LPH-33) to 7.3 nM (8) and 3.1 nM (13). These compounds are the first-in-class LpxH inhibitors that achieve chelation of the di-manganese cluster in LpxH.

### Example 2—MIC Assay

The MIC assay protocol was adapted from the broth microdilution methods described by the National Committee for Clinical Laboratory Standards using 96-well plates as described previously. In brief, overnight bacterial cultures were diluted to an OD₆₀₀ of 0.006 in cation-adjusted Mueller-Hinton medium containing 7% DMSO and incubated at 37° C. for 22 h in the presence of varying concentrations of inhibitors. Following overnight incubation, each well is treated with 10 μL of 3-(4,5-dimethylthiazol-2-yl)-2,5-diphenyltetrazolium bromide (MTT, 5 mg/mL) for 3 h. Finally, the culture is solubilized by addition of 2-propanol, and the results are read by taking the difference of the UV reading at both 570 and 690 nm. MIC values were reported as the lowest compound concentration that inhibited bacterial growth. The baseline for inhibited cell growth was determined to be <4x the average control reading due to the baseline optical density of the initial starting bacterial culture.

MIC values of some of the compounds of Table 1 is shown in Table 4.

TABLE 4

| | MIC (mg/L) - K. pneumoniae | MIC (mg/L) - E. coli |
|---|---|---|
| Drug | (ATCC 10031) | W3110 |
| AZ1 | >64 | — |
| JH-LPH-28 | 3 | — |
| JH-LPH-33 | 0.83 | — |
| JH-LPH-38 | — | — |
| JH-LPH-39 | — | — |
| JH-LPH-41 | 10.67 | — |
| JH-LPH-42 | >64 | — |
| JH-LPH-43 | — | — |
| JH-LPH-44 | — | — |
| JH-LPH-45 | 18.67 | — |
| JH-LPH-46 | — | — |
| JH-LPH-47 | >64 | — |
| JH-LPH-48 | >64 | — |
| JH-LPH-49 | 18.67 | — |
| JH-LPH-50 | 3.33 | — |
| JH-LPH-54 | — | — |
| JH-LPH-55 | — | — |
| JH-LPH-59 | — | — |
| JH-LPH-56 | — | — |
| JH-LPH-60 | — | — |
| JH-LPH-61 | >64 | — |
| JH-LPH-62 | 24 | — |
| JH-LPH-64 | 20 | — |
| JH-LPH-65 | 12 | — |
| JH-LPH-66 | 26.67 | — |
| JH-LPH-67 | 28 | — |
| JH-LPH-68 | 28 | — |
| JH-LPH-69 | 4 | — |
| JH-LPH-70 | 10.67 | — |
| JH-LPH-71 | 8 | — |
| JH-LPH-72 | >64 | — |
| JH-LPH-73 | 0.67 | — |
| JH-LPH-74 | >64 | — |
| JH-LPH-75 | >64 | — |
| JH-LPH-76 | >64 | — |
| JH-LPH-77 | >64 | — |
| JH-LPH-78 | — | — |
| JH-LPH-79 | — | — |
| JH-LPH-80 | — | — |
| JH-LPH-81 | 32 | — |
| JH-LPH-82 | >64 | — |
| JH-LPH-83 | 64 | — |
| JH-LPH-84 | 32 | — |
| JH-LPH-85 | 12 | — |
| JH-LPH-86 | 0.125 | — |
| JH-LPH-87 | >64 | — |
| JH-LPH-88 | 64 | — |
| JH-LPH-89 | — | — |
| JH-LPH-90 | 2 | — |
| JH-LPH-91 | 0.125 | — |
| JH-LPH-92 | 0.063 | — |
| JH-LPH-93 | 1.33 | — |
| JH-LPH-94 | >64 | — |
| JH-LPH-95 | 4 | — |
| JH-LPH-96 | 0.83 | — |
| JH-LPH-97 | 0.10 | — |
| JH-LPH-98 | — | — |
| JH-LPH-99 | — | — |
| JH-LPH-100 | — | — |
| JH-LPH-101 | — | — |
| JH-LPH-102 | — | — |
| JH-LPH-103 | — | — |
| JH-LPH-104 | 1.0 | 16.0 |
| JH-LPH-105 | 0.3 | 2.0 |
| JH-LPH-106 | 0.04 | 1.7 |
| JH-LPH-107 | 0.04 | 1.0 |

### Example 2—Enzymatic Assay for KpLpxH Inhibition

The LpxE-coupled LpxH activity assay was conducted as described previously using the GB1-K. pneumoniae LpxH-His₁₀ fusion protein. Briefly, two reaction mixtures were prepared. Mixture 1 contains 20 mM Tris-HCl (pH 8.0), 0.5 mg/mL BSA, 0.02% Triton X-100, 1 mM MnCl₂, 1 mM OTT, 10% DMSO, and 200 μM substrate (UDP-DAGn), and mixture 2 is comprised of the same buffer, but instead of substrate, contains both LpxH (20 ng/mL) and 2X inhibitor. These mixtures were then pre-incubated at 37° C. for 10 min. To initiate the reaction, an equal volume of the LpxH mixture (mixture 2) was added to the substrate mixture (mixture 1) at 37° C. The final reaction solution contains 100 μM substrate, 10 ng/mL enzyme, and 1 x inhibitor. At the desired reaction time points, an aliquot of 20 μL reaction mixture was removed and added to a well in 96-well half-area plate containing 5 mM EDTA (final concentration) to quench the LpxH reaction. Purified Aquifex aeolicus LpxE was then added to a final concentration of 5 μg/mL. The plate was incubated at 37° C. for 30 min followed by addition of formic acid to a final concentration of 3.75 M to quench the LpxE reaction. The malachite green reagent (Sigma Aldrich, catalog MAK307) was diluted 5-fold into the solutions, and the plate was incubated for 30 min at room temperature before the absorbance at 620 nm was measured. All measurements were done in triplicates, and standard error (S.E.) was calculated.

IC₅₀ of compounds of Table 1 is shown in Table 5.

TABLE 5

| | IC₅₀: Average | IC₅₀: SEM |
|---|---|---|
| Compound | (micoM) | (microM) |
| AZ1 | 0.361 | 0.020 |
| JH-LPH-28 | 0.139 | 0.021 |
| JH-LPH-33 | 0.104 | 0.011 |
| JH-LPH-41 | 0.073 | 0.006 |
| JH-LPH-45 | 0.017 | 0.002 |
| JH-LPH-48 | 0.049 | 0.006 |
| JH-LPH-49 | 0.152 | 0.002 |
| JH-LPH-50 | 0.008 | 0.001 |
| JH-LPH-65 | 0.013 | 0.003 |
| JH-LPH-69 | 0.079 | 0.011 |
| JH-LPH-70 | 0.062 | 0.010 |
| JH-LPH-73 | 0.037 | 0.002 |
| JH-LPH-79 | 0.037 | |
| JH-LPH-80 | 0.431 | 0.025 |
| JH-LPH-86 | 0.085 | 0.012 |
| JH-LPH-88 | 3.204 | 0.416 |
| JH-LPH-89 | 2.437 | 0.273 |
| JH-LPH-90 | 0.111 | 0.011 |
| JH-LPH-91 | 0.105 | 0.015 |
| JH-LPH-92 | 0.005 | 0.001 |
| JH-LPH-93 | 0.032 | |
| JH-LPH-96 | 0.002 | 0.00004 |
| JH-LPH-97 | 0.008 | 0.00034 |
| JH-LPH-98 | 0.024 | 0.00572 |
| JH-LPH-99 | 0.011 | 0.00241 |
| JH-LPH-104 | 0.00023 | 0.00005 |

1. A compound of formula (I), or a pharmaceutically acceptable salt thereof: 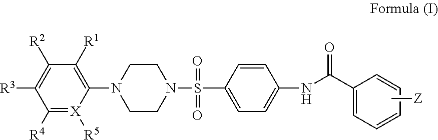
wherein
X is carbon or nitrogen;
R¹-R⁵ are each independently hydrogen, hydroxyl, halogen, CF₃, C1-C4 alkyl, or substituted C1-C4 alkyl, or absent, wherein substituted C1-C4 alkyl is optionally halogen substituted C1-C4 alkyl; and
Z is hydrogen, an alkylsulfonamide, or a substituted alkylsulfonamide attached to the aryl by either N or S.

2. The compound of claim 1, wherein the halogen is fluorine, chlorine, or bromine.

3. The compound of claim 1, wherein the halogen is chorine.

4. The compound of claim 1, wherein R² and R⁴ are independently halogen or CF₃.

5. The compound of claim 1, wherein R² and R⁴ are independently chlorine or CF₃.

6. The compound of claim 1, wherein R¹ and R³ are hydrogen, X is nitrogen, and R⁵ is absent.

7. The compound of claim 1, wherein R¹ and Rt³ are hydrogen.

8. The compound of claim 1, wherein X is nitrogen and R⁵ is absent.

9. The compound of claim 1, wherein X is carbon and R⁵ is hydrogen.

10. A compound of formula (II), or a pharmaceutically acceptable salt thereof: 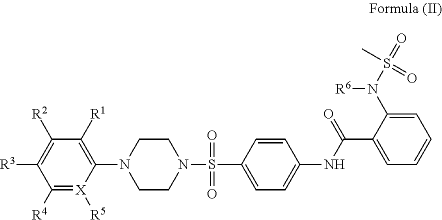
X is carbon or nitrogen,
R¹-R⁶ are each independently hydrogen, hydroxyl, halogen, CF₃, C1-C4 alkyl, or substituted C1-C4 alkyl, or absent, wherein substituted C1-C4 alkyl is optionally halogen substituted C1-C4 alkyl.

11. (canceled)

12. The compound of claim 10, wherein the halogen is chlorine.

13. The compound of claim 10, wherein R² and R⁴ are independently halogen or CF₃.

14. (canceled)

15. (canceled)

16. The compound of claim 10, wherein R¹ and R³ are hydrogen.

17. The compound of claim 10, wherein X is nitrogen and R⁵ is absent.

18. The compound of claim 10, wherein X is carbon and R⁵ is hydrogen.

19. (canceled)

20. (canceled)

21. The compound of claim 10, wherein the compound is

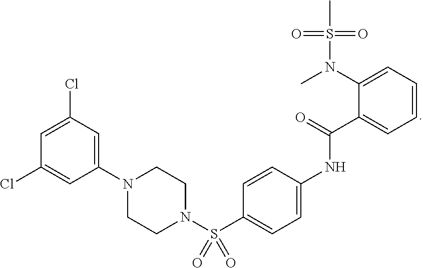 [SMILES: CN(c1ccccc1C(=O)Nc1ccc(S(=O)(=O)N2CCN(c3cc(Cl)cc(Cl)c3)CC2)cc1)S(C)(=O)=O]

22. A compound of formula (III), or a pharmaceutically acceptable salt thereof: 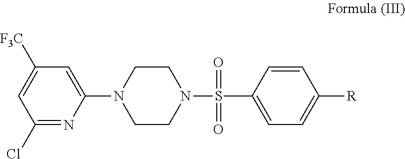
wherein
R is a substituted amide with formula —NHC(O)A; and
A is a substituted aryl.

23. (canceled)

24. (canceled)

25. (canceled)

26. (canceled)

27. A compound of formula (IV), or a pharmaceutically acceptable salt thereof: 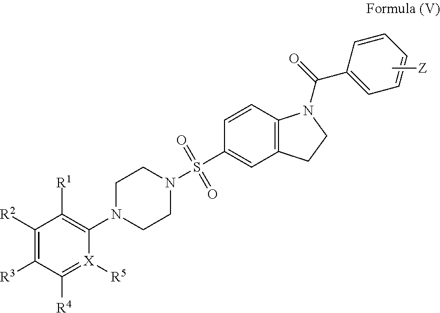
wherein
X is carbon or nitrogen;
R¹-R⁵ are each independently hydrogen, hydroxyl, halogen, CF₃, C1-C1 alkyl, or substituted C1-C4 alkyl, or absent, wherein substituted (C1-C4 alkyl is optionally halogen substituted C1-C4 alkyl; and
wherein Z is hydrogen, an alkylsulfonamide, or a substituted alkylsulfonamide attached to the aryl by either N or S.

28. (canceled)

29. (canceled)

30. The compound of claim 27, wherein R² and R⁴ are independently halogen or CF₃.

31. (canceled)

32. (canceled)

33. (canceled)

34. (canceled)

35. (canceled)

36. The compound of claim 27, wherein the compound is

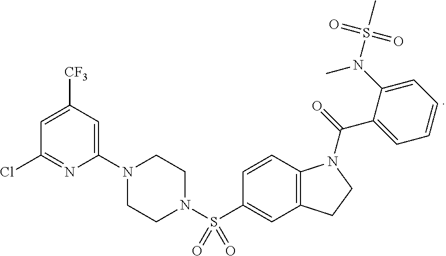 [SMILES: CN(c1ccccc1C(=O)N1CCc2cc(S(=O)(=O)N3CCN(c4cc(C(F)(F)F)cc(Cl)n4)CC3)ccc21)S(C)(=O)=O]

37. A compound, or a pharmaceutically acceptable salt thereof wherein the compound is selected from the group consisting of:

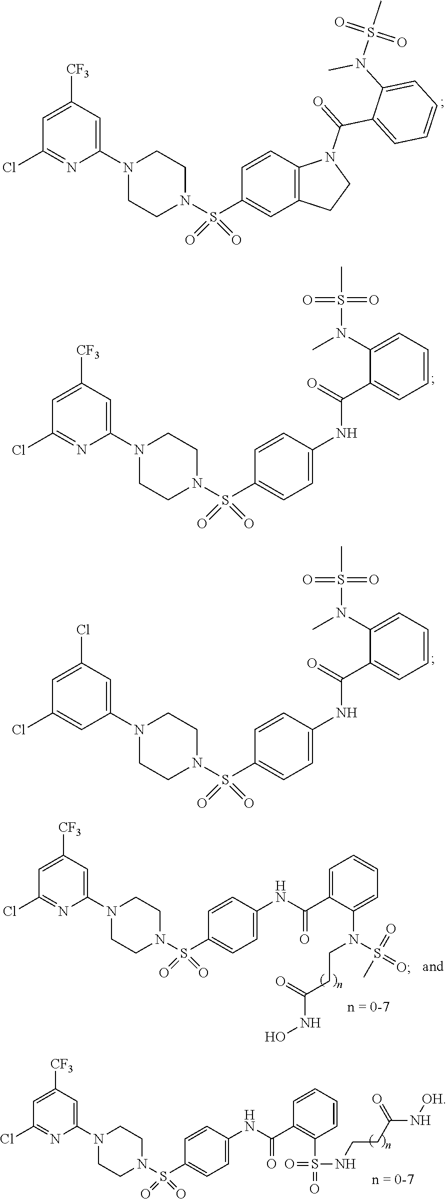 [SMILES: CN(c1ccccc1C(=O)N1CCc2cc(S(=O)(=O)N3CCN(c4cc(C(F)(F)F)cc(Cl)n4)CC3)ccc21)S(C)(=O)=O ; CN(c1ccccc1C(=O)Nc1ccc(S(=O)(=O)N2CCN(c3cc(C(F)(F)F)cc(Cl)n3)CC2)cc1)S(C)(=O)=O ; CN(c1ccccc1C(=O)Nc1ccc(S(=O)(=O)N2CCN(c3cc(Cl)cc(Cl)c3)CC2)cc1)S(C)(=O)=O ; CS(=O)(=O)N(CCC(=O)NO)c1ccccc1C(=O)Nc1ccc(S(=O)(=O)N2CCN(c3cc(C(F)(F)F)cc(Cl)n3)CC2)cc1 ; O=C(CCNS(=O)(=O)c1ccccc1C(=O)Nc1ccc(S(=O)(=O)N2CCN(c3cc(C(F)(F)F)cc(Cl)n3)CC2)cc1)NO]

38. (canceled)

39. A pharmaceutical composition comprising the compound of claim 1 and a pharmaceutically acceptable carrier, diluent, and/or an excipient.
